SEQUENCE LISTING (4 sequences)

SEQ ID NO: 1 - DNA, 3109 bp, Homo sapiens
  CDS (15)..(1556)
cattggacttcagcatgactactcagttgccagcttacgtggcaattttgcttttctatgtctcaagagccagctgccaggacactttcattgcagctgtttatgagcatgcagcgatattgcccaatgccaccctaacaccagtgtctcgtgaggaggctttggcattaatgaatcggaatctggacattttggaaggagcgatcacatcagcagcagatcagggtgcgcatattattgtgactccagaagatgctatttatggctggaacttcaacagggactctctctacccatatttggaggacatcccagaccctgaagtaaactggatcccctgtaataatcgtaacagatttggccagaccccagtacaagaaagactcagctgcctggccaagaacaactctatctatgttgtggcaaatattggggacaagaagccatgcgataccagtgatcctcagtgtccccctgatggccgttaccaatacaacactgatgtggtatttgattctcaaggaaaactggtggcacgctaccataagcaaaaccttttcatgggtgaaaatcaattcaatgtacccaaggagcctgagattgtgactttcaataccacctttggaagttttggcattttcacatgctttgatatactcttccatgatcctgctgttaccttggtgaaagatttccacgtggacaccatagtattcccaacagcttggatgaatgttttgccacatttgtcagctgttgaattccactcagcttgggctatgggcatgagggtcaatttccttgcatccaacatacattacccctcaaagaaaatgacaggaagtggcatctatgcacccaattcttcaagagcatttcattatgatatgaagacagaagagggaaaactcctcctctcgcaactggattcccacccatcccattctgcagtggtgaactggacttcctatgccagcagtatagaagcgctctcatcaggaaacaaggaatttaaaggcactgtctttttcgatgaattcacttttgtgaagctcacaggagttgcaggaaattatacagtttgtcagaaagatctctgctgtcatttaagctacaaaatgtctgagaacataccaaatgaagtgtacgctctaggggcatttgacggactgcacactgtggaagggcgctattatctacagatttgtaccctgttgaaatgtaaaacgactaatttaaacacttgcggtgactcagctgaaacagcttctaccaggtttgaaatgttctccctcagtggcactttcggaacccagtatgtctttcctgaggtgttgctgagtgaaaatcagcttgcacctggagaatttcaggtgtcaactgacggacgcttgtttagtctgaagccaacatccggacctgtcttaacagtaactctgtttgggaggttgtatgagaaggactgggcatcaaatgcttcatcaggcctcacagcacaagcaagaataataatgctaatagttatagcacctattgtatgctcattaagttggtagaatattgactttttctcttttttatttgggataatttaaaaaatgatggatgagaaaagaaagattggtccgggttaatattatcctctagtataagtgaattactagtttctctttatttagacaaacacacacacaccagataatataaacttaataaattatctgttaatgtagattttatttaaaaaactatatttgaacattggtctttcttggacgtgagctaattatatcaaataagtatcacaaatcttttacgcagaagaaataaaaactacgggtagaaaacataagaactatcataaaatttacttacaaggaggctgctcttgttaccacttttattatattacgtatcacttattcagctctgctgaaaatttccaatgactttgtttgtttgctcttttagttttttacctaaacaatacattttgattctcttgtgggttgataatgtctccccaaaatttacatgttgaagcacctcagaatgtgactgtatttggagacagggtctttaaagaggtaaaataaggtcattaggatagaccctaattcaatatgactgatgatcataaaagaagaggcgagtagggcacaacaggcacaaagggagaccataaggagacacagaggaaggacaactctttacaagctaagaagagagggcctcagaagaaaccaaccctgccaacaccttgatcttggacttccagcctccaaaactatgagaaataaatttctattgtttaagtcacccagtccatggtactttgttaggcagccctggcaaatgaatcaaagacccattcctgttcctctccccaccactactgttttctactgtaatctgaagcttcaacaaaaggcttacctggtaagaatattcagctggtctgggtcctcaagactccaatagacactcttaaagaaggattgctgatggattgatagtgaaaccattagatcattgaattcctctggaattagaaaaccagagagtcccattttaagaaattagatatttaatatagcattgtgtgttctattttagtaacagcagaatctcttgacattacacaactcagtgaaacaacatcatttaagccaaaatatctcccaactgactgatagactctgagcactaatatcatagtgctgtgatgatggacaattacatagtaccgataacagccatgcactgtgcaaagcatgcccttctgcacaggagagcaaggcacttgcagtagtgatctatgccagcaaaacatcattttgagacaaacatttttgtggcagatgtttttcctaaaaagtactatatcatccaagaaatatttgagtaaaatcccttgttcttttgggtgacattaactgacatttgctttttttcaagacctaatagaaaataagaaagcccataatgtatttagaaacaggaatcctcagagcaattctctgtattctcatataatttcaatgtaaaacagaaaacatattgatgtgttggtgataggcttgaattattaaaaacttcaaaaacaaaa

SEQ ID NO: 2 - protein, 513 aa, Homo sapiens
MTTQLPAYVAILLFYVSRASCQDTFIAAVYEHAAILPNATLTPVSREEALALMNRNLDILEGAITSAADQGAHIIVTPEDAIYGWNFNRDSLYPYLEDIPDPEVNWIPCNNRNRFGQTPVQERLSCLAKNNSIYVVANIGDKKPCDTSDPQCPPDGRYQYNTDVVFDSQGKLVARYHKQNLFMGENQFNVPKEPEIVTFNTTFGSFGIFTCFDILFHDPAVTLVKDFHVDTIVFPTAWMNVLPHLSAVEFHSAWAMGMRVNFLASNIHYPSKKMTGSGIYAPNSSRAFHYDMKTEEGKLLLSQLDSHPSHSAVVNWTSYASSIEALSSGNKEFKGTVFFDEFTFVKLTGVAGNYTVCQKDLCCHLSYKMSENIPNEVYALGAFDGLHTVEGRYYLQICTLLKCKTTNLNTCGDSAETASTRFEMFSLSGTFGTQYVFPEVLLSENQLAPGEFQVSTDGRLFSLKPTSGPVLTVTLFGRLYEKDWASNASSGLTAQARIIMLIVIAPIVCSLSW

SEQ ID NO: 3 - DNA, 2255 bp, Mus musculus
  CDS (22)..(1560)
ttgctgtcgttggacttcagcatgggcacgtcttggtggctggcgtgtgctgcagcgttttctgccctctgtgtcttaaaagccagctcgctggatactttcctcgcggctgtttacgagcatgctgtgatcctgcctaaggacaccctgttgccagtgtctcacggtgaggctctggcattaatgaaccagaatctggaccttctggaaggagcgatcgtatctgcagcgaagcagggtgcgcacattattgtgactccagaagatggcatatacggtgtgcgtttcaccagggatacgatctacccatacctggaggagatcccagaccctcaagtaaactggataccctgtgataaccctaaaagatttggctctaccccggtgcaggagagactcagctgcttggccaagaacaactccatctatgttgtggcgaacatgggagacaagaagccgtgtaacaccagcgactctcactgtccacctgacggcagattccagtacaacactgatgtggtgtttgattcccagggtaaactggttgcgagataccataagcaaaacattttcatgggagaagatcagttcaatgtccccatggagcctgagtttgtgactttcgacaccccctttggaaagtttggcgtcttcacctgtttcgatattctcttccatgatcccgctgtcaccctggtgacagaattccaggtggacaccatactgttcccaaccgcctggatggacgtccttcctcatttggcagccattgaattccactcagcttgggctatgggcatgggggtcaatttcctagcagctaatctacataatccctcgaggagaatgacaggaagtggtatctatgcacccgattctccaagggtctttcactacgacaggaagacccaagaaggaaaactcctcttcgctcagctgaaatcccacccaattcactccccggtgaactggacttcctatgctagcagtgtagaatcaaccccaaccaaaacccaggaatttcagagtattgtcttttttgatgagtttacctttgtggagctcaaagggatcaaaggaaattacactgtttgccagaatgacctctgctgtcacctaagctaccagatgtctgagaagcgagcagatgaggtttatgcctttggagcctttgatgggctgcacaccgtggaagggcagtactacctacagatctgcatcctgctaaaatgtaaaactaccaatttacgcacctgtggtagttcagtggacacggcttttaccaggtttgaaatgttctcgctcagcggcacttttggaacccggtatgtcttccctgaagtgttgctgagtgaggtcaagctcgcacctggggagtttcaggtgtcaagtgatgggcgcctggttagcctgaagccaacctcgggacctgtgttaaccatcgggctctttgggaggttgtatgggaaggactgggcatccaatgcttcctcagacttcatagcacactcgctgataataatgctgattgtgacgcctattatacattacttgtgctgatggaatttttacattttttattttatttagaaaatttaaaattggtggatgcagaaaaaataactgtttgtcaacagtggactcgggtgtaagcaaataaagtgcctcttctttagaaaaacatatgtacaccagatacatttcaggaaaattaataaaactttgagcattggaacgagatggagggccaagtaaaggtcgcatgtgttttattcagaagaaataaaaattacagttaaaaggcacttcaaaccatcataagatagatttacaagaggtgtaaatctattatacatcttactcagttatgcttagaatttccaatgtgtttgttcatttgggctattaagtatttatctcaacatttccgttctctcatggaccagatcctgtagttttaattcttcagttcaagtcccagttcccacaacctcagaacgtgactgccttggtgtctttggcaatgaagacataagaggcatcattagcatggactttaattcaatatgactgatctcctcagaagaaatcaggacaaagacttgcatcaagtgaagcccttgtgaacacaggaaaagatggtcatgtacaacaagaaaaggggcctcaggagaacgcaaacctgctaacgtgtcaaacttccaggtctccagaatcatgaggcaataaatttctgttttaaatgaaaaaaaaaa

SEQ ID NO: 4 - protein, 512 aa, Mus musculus
MGTSWWLACAAAFSALCVLKASSLDTFLAAVYEHAVILPKDTLLPVSHGEALALMNQNLDLLEGAIVSAAKQGAHIIVTPEDGIYGVRFTRDTIYPYLEEIPDPQVNWIPCDNPKRFGSTPVQERLSCLAKNNSIYVVANMGDKKPCNTSDSHCPPDGRFQYNTDVVFDSQGKLVARYHKQNIFMGEDQFNVPMEPEFVTFDTPFGKFGVFTCFDILFHDPAVTLVTEFQVDTILFPTAWMDVLPHLAAIEFHSAWAMGMGVNFLAANLHNPSRRMTGSGIYAPDSPRVFHYDRKTQEGKLLFAQLKSHPIHSPVNWTSYASSVESTPTKTQEFQSIVFFDEFTFVELKGIKGNYTVCQNDLCCHLSYQMSEKRADEVYAFGAFDGLHTVEGQYYLQICILLKCKTTNLRTCGSSVDTAFTRFEMFSLSGTFGTRYVFPEVLLSEVKLAPGEFQVSSDGRLVSLKPTSGPVLTIGLFGRLYGKDWASNASSDFIAHSLIIMLIVTPIIHYLC

(19) United States
(12) Patent Application Publication
(10) Pub. No.: US 2004/0247524 A1
(43) Pub. Date: Dec. 9, 2004

(54) Method of treatment of an inflammatory disorder with a Vanin-1 antagonist

(76) Inventors: Philippe Naquet, Marseille (FR); Franck Galland, Marseille (FR); Florent Martin, San Diego, CA (US); Max De Reggi, Marseille (FR); Bouchra Di Reggi, Marseille (FR); Marie-France Penet, Marseille (FR); Giuseppina Pitari, L'Aquila (IT); Sylvestro Dupre, Roma (IT)

Correspondence Address: STITES & HARBISON PLLC, 1199 NORTH FAIRFAX STREET, SUITE 900, ALEXANDRIA, VA, 22314, US

(21) Appl. No.: 10/454,822

(22) Filed: Jun. 5, 2003

(51) Int. Cl. A61K 49/00

(52) U.S. Cl. 424/9.2

(57) ABSTRACT

The present invention relates to the screening of Vanin-1 antagonists useful for the treatment of an inflammatory disorder, in particular intestinal inflammation, and to a method treatment of an inflammatory disorder with such a Vanin-1 antagonist.

Cysteamine, a low molecular weight thiol (Miller, 1993), is believed to be involved in enzymatic regulation via sulfhydryl-disulfide exchange reactions between sulfhydryl groups of enzymes and the oxidized form cystamine (Ernest, 1973; Pontremoli, 1967; Namboodiri, 1980; Siefring, 1978). One of the enzymes putatively inhibited by cysteamine is γ-glutamylcysteine synthetase (γ-GCS) which catalyses the first step of glutathione (GSH) synthesis (Seelig, 1984; Griffith, 1977; Lebo, 1978 Beamer, 1980). GSH is an important intracellular tripeptide with multiple functions including redox regulation in inflammation (Haddad, 2002). In vivo, the concentration of cysteamine in tissue is normally low and is in equilibrium with its oxidized form cystamine (Ricci, 1983). Cystoamine is produced upon hydrolysis of pantetheine (Dupre, 1975) by the action of a specific enzymatic activity called pantetheinase (EC 3.5.1.-), with the, concomitant release of pantothenic acid (vitamin B5). In mouse this relatively ubiquitous enzyme is encoded by two genes (Maras, 1999; Galland, 1998; Granjeaud, 1999), Vanin-1 and Vanin-3, preferentially expressed by epithelial and myeloid cells, respectively (Martin, 2001). In human and drosophila, this enzyme is encoded by 3 genes (VNN-1, VNN-2, VNN-3). In mouse and human, Vanin-1 and VNN1, respectively, are GPI-anchored to cell membranes and are highly expressed at the brush border of various epithelial cells including intestinal enterocytes, kidney tubular cells, hepatocytes, pancreatic acinar cells, thymic medullary epithelial cells (Galland, 1998; Aurrand-Lions, 1996; Pitari, 2000; Martin, 2001). In drosophila, 4 genes homologous to the mammalian Vanin sequences are identified and preliminary studies show that drosophila has a pantetheinase activity (Granjeaud et al, 1999).

Vanin-1 deficient (Vanin-1^{-/-}) mice develop normally but have no detectable free cyst(e)amine in kidney and liver, in spite of the presence of Vanin-3 (Pitari, 2000). Therefore they offer the unique opportunity to explore the in vivo function of cysteamine which is poorly characterised.

Schistosomiasis is characterised by an altered balance between pro- and anti-oxidant processes (Abdallahi, 1999; La Flamme, 2001; Gharib, 1999; Pascal, 2000), leading to hepatic and intestinal pathology (Lambertucci, 2000). Similarly, NSAID (non-steroidal anti-inflammatory drug)-induced damage is at least in part associated with oxidative stress, since gp91^{phox-/-} mice that lack NADPH oxidase activity are less susceptible to NSAID injury than wild-type (WT) mice (Beck, 2000).

The inventors investigated the response of Vanin-1^{-/-} mice to chronic and acute intestinal inflammation triggered by Schistosoma mansoni (S. mansoni) infection and treatment with high doses of the non-steroidal anti-inflammatory drug indomethacin, respectively.

The inventors demonstrated that inactivation of the Vanin-1 gene prevents acute and chronic inflammation since in both cases intestinal injury was moderate in Vanin-1 deficient mice, as compared to controls. The protection was associated with reduced expression of inflammatory molecules, myeloid cell recruitment and mucosal damage in the intestine. Furthermore, glutathione synthesis and storage were increased in liver and intestine.

These events were further shown to be associated with the lack of free cysteamine/cystamine, which is undetectable in Vanin-1 deficient mice, since cystamine given orally reversed the inflammatory phenotype. This reverting effect was correlated with inhibition of glutathion synthesis in vivo.

Furthermore, since Vanin-1 was initially described as involved in thymus homing (Aurrand-Lions, 1996), the inventors investigated mechanisms involved in the regulation of thymic reconstitution following sublethal ionizing irradiation of Vanin1^{-/-} mice. In the absence of Vanin-1, reconstitution of thymus following irradiation was found to be accelerated 2 to 3 fold, as compared to wild-type mice. These results show that the Vanin/cysteamine system is involved in the regulation of the response to oxidative stress.

Thus, these results provided by the inventors show that inhibition of pantetheinase/Vanin-1 activity provides a new strategy for the treatment of inflammatory disorders and/or oxidative processes, in particular intestinal inflammation.

### Definitions

As used herein, the term “Vanin-1” denotes a human protein (VNN-1) having a pantetheinase activity (EC 3.5.1.-) and described in Genebank under the accession number NM_{—}004666 (DNA sequence: SEQ ID No 1, amino acid sequence: SEQ ID No 2), but also homologous proteins from other species, in particular mammalian species such as mouse (Genebank accession number: NM_{—}011704, DNA sequence: SEQ ID No 3, amino acid sequence: SEQ ID No 4), rat, pig, monkey.

The term “Vanin-1 antagonist” is defined herein as a compound which, in vitro and/or in vivo: (i) inhibits the activity and/or expression of Vanin-1; and/or (ii) blocks processing of pantetheine into cysteamine and pantothenic acid; and/or (iii) blocks intracellular synthesis of cysteamine and/or of cystamine, the oxidized form of cysteamine. Inhibition and blocking may be total or partial.

As used herein, “inflammatory disorder” denotes a condition of sustained or chronic inflammation that occurs when tissues are injured by viruses, bacteria, trauma, chemicals, heat, cold or any other harmful stimulus. Preferably, an inflammatory disorder according to the invention is a gastrointestinal inflammatory disorder that may be selected from the group consisting of an inflammatory bowel disease (IBD) such as Irritable Bowel Syndrome (IBS), ulcerative colitis and Crohn's disease, an ulcer resulting from administration of a non-steroidal anti-inflammatory drug, such as a peptic ulcer (i.e. a sore that forms in the lining of the stomach or the duodenum), and an inflammatory disorder associated with an infection with Schistosoma mansoni parasite.

The term “non-steroidal anti-inflammatory drugs” or “NSAID” is meant for non-steroidal molecules that are generally administered for the treatment of an inflammatory disorder such as rheumatoid arthritis, for instance. Examples of non-steroidal anti-inflammatory drugs include detoprofen, diclofenac, diflunisal, etodolac, fenoprofen, flurbiprofen, ibuprofen, indomethacin, ketoprofen, meclofenameate, mefenamic acid, meloxicam, nabumeone, naproxen sodium, oxaprozin, piroxicam, sulindac, tolmetin, celecoxib, rofecoxib, aspirin, choline salicylate, salsalte, and sodium and magnesium salicylate. However, administration of a NSAID to a patient may be associated with the development of an ulceration of the digestive tract, in particular stomach ulceration and/or intestinal inflamrriation.

The term “oxidative disorder” denotes troubles induced by an oxidative stress (for instance irradiation such as gamma irradiation, intoxication by xenoblotics, infection, administration of a compound such as ter-butyl-hydroquinone (TBHQ), or peroxide hydrogen (H₂O₂)), i.e. a disturbance in the prooxidant-antioxidant balance in favour of the prooxidant, leading in general to cell or tissue damage (for instance ulcerations, mutagenesis, apoptosis, fibrosis) and in most cases acute or chronic inflammation (for instance post-irradiation colitis, lung, liver or kidney fibrosis). This imbalance may be due to either an over-production of free radical oxygen species (ROS) or a deficiency in the cellular antioxidant defense. Prolonged high levels of ROS can cause damage to all biological macromolecules and play a role in acute and chronic cell injury. Therefore, cells in injured and inflamed tissues must be able to protect themselves against ROS toxicity. In normal conditions oxidative lesions can be repaired by free radical scavengers and a series of enzymes such as superoxide dismutase (SOD), catalase and glutathione peroxidases (GPXs). Glutathione (GSH) is a non-protein sulfhydryl compound that plays a prominent role as an intracellular protectant, acting as a radical scavenger but also a co-factor for detoxifying enzymes such as GPXs and glutathione S-transferases (GSTs). In addition, cellular antioxidant mechanisms include the coordinate induction of a number of genes that encodes detoxifying enzymes and oxidative stress-induced proteins via antioxidant response elements (AREs) in their promoter region.

At the cellular level, the inflammatory or oxidative disorders are reflected by an inflammatory or oxidative state. This state may be assessed by markers of oxidative stress which may be, but not exclusively i) alterations in GSH stores, detoxication enzymes (for instance SOD, catalase), GSH-dependent enzymatic activities (glutathion peroxidases, glutathion transferases); ii) activation of stress pathways (for instance MAPK and JNK kinases, NF-kB and API transcription factors; iii) production of inflammatory lipids (for instance prostaglandins and leucotrienes); iv) cell and molecular lesions (for instance among which protein oxidation, nitrosilation and carbonylation, lipid oxidation, DNA mutations).

The term “test compound” is intended for a molecule of known or unknown structure, or a mixture of molecules, or an extract of a natural product. Preferably, the test compound has a Vanin-I antagonist activity.

As used herein, the term “mammal” includes a human and non-human mammals, including rodents (e.g. a rat, a mouse or a rabbit), monkeys, cattle, sheep, feline (e.g. a cat), canine (e.g. a dog), horses, goats . . .

In the context of the invention, the term “treating” or “treatment” is meant the prophylactic or curative treatment of a disorder, i.e. reversing, alleviating, inhibiting the progress of, or preventing the disorder or condition to which such term applies, or one or more symptoms of such disorder or condition. The treatment may be associated with another pre-existing treatment in order to improve the efficacy of said pre-existing treatment.

### Vanin-1 Antagonists

Said Vanin-1 antagonist may be a direct inhibitor of Vanin-1 activity.

Such an inhibitor may be any peptide, peptidomimetics or non peptidic mimetics (Rubin-Carrez, 2000), such as small organic molecules capable of interfering with the pantheteinase activity of Vanin-1, e.g. through blocking processing of pantetheine into cysteamine and pantothenic acid, and/or blocking intracellular synthesis of cysteamine and/or cystamine.

Inhibitors can be readily identified by screening methods, including biochemical and cellular in vitro assays. In particular a test compound may be assayed for its ability to inhibit Vanin-1 activity by contacting a cell that expresses Vanin-1 and testing the ability of the test compound to inhibit Vanin-1 activity, i.e. to block or decrease cellular synthesis; of cysteamine, and/or to modulate GSH-dependent enzymatic activities or transduction/transcription pathways. The decrease in the level of cysteamine and/or cystamine synthesis in comparison with a Vanin-1 expressing cell that was not subjected to this test compound, is indicative of a substance that shows inhibiting activity toward Vanin-1. For instance, such a test compound may be an artificial pantetheinase substrates (Pitari, 1994, 2000; Dupré, 1970).

Alternatively, a process for assaying compounds for their ability to inhibit Vanin-1 activity may comprise the steps of providing Vanin-1, for instance recombinant Vanin-1, and testing the ability of the candidate substances to inhibit Vanin-1 activity, i.e. to block or decrease processing of pantetheine into cysteamine and pantothenic acid. The decrease in the level of cysteamine synthesis, or a decrease in pantetheine processing, in comparison with pantetheine not subjected to this test compound, is indicative of a substance that shows inhibiting activity toward Vanin-1. Kinetic studies of pantetheinase inhibition have been described for instance in Pitari et al (1994).

Examples of Vanin-1 inhibitors include compounds such as described by Pitari et al. (1994): non specific alkylating agents (leading to irreversible inhibition) such as iodoacetamide, iodoacetate, N-ethyl maleimide, and/or disulfides (leading to a reversible inhibition) such as pantethine (the oxidized form of pantetheine), cystamine, 5,5′-dithiobis(2-nitrobenzoic acid), 4,4′-dithiodipyridine and oxidized mercaptoethanol. So far, one considers the pantothenate part of the pantetheine molecule to be recognised specifically by pantetheinase; so a typical inhibitory compound may contain this moiety hooked to a non hydrolysable bond on the C-end of the molecule.

The Vanin-1 antagonist may also be a monoclonal or polyclonal antibody, or a fragment thereof, or a chimeric or immunoconjugate antiboby, which is capable of specifically interacting with Vanin-1 and inhibiting its activity (blocking antibody). Alternatively, said inhibitor may be capable of interacting with Vanin-1 substrate, i.e. pantheteine, thereby blocking processing of pantetheine into cysteamine. The antibodies of the present invention can be single chain or double chain, chimeric antibodies, humanized antibodies, or portions of an immunoglobulin molecule, including those portions known in the art as antigen binding fragments Fab, Fab′, F(ab′)2 and F(v). They can also be immunoconjugated,e.g. with a toxine, or labelled antibodies.

Whereas polyclonal antibodies may be used, monoclonal antibodies are preferred since they are more reproducible in the long run.

Procedures for raising polyclonal antibodies are also well known. Polyclonal antibodies can be obtained from serum of an animal immunized against Vanin-1 or pantetheine, which can be produced by genetic engineering for example according to standard methods well-known by one skilled in the art. Typically, such antibodies can be raised by administering the protein subcutaneously to New Zealand white rabbits which have first been bled to obtain pre-immune serum. The antigens can be injected at a total volume of 100 μl per site at six different sites. Each injected material will contain adjuvants with or without pulverized acrylamide gel containing the protein or polypeptide after SDS-polyacrylamide gel electrophoresis. The rabbits are then bled two weeks after the first injection and periodically boosted with the same antigen three times every six weeks. A sample of serum is then collected 10 days after each boost. Polyclonal antibodies are then recovered from the serum by affinity chromatography using the corresponding antigen to capture the antibody. This and other procedures for raising polyclonal antibodies are disclosed in Harlow et al. (1988) which is hereby incorporated by reference.

A “monoclonal antibody” in its various grammatical forms refers to a population of antibody molecules that contain only one species of antibody combining site capable of immunoreacting with a particular epitope. A monoclonal antibody thus typically displays a single binding affinity for any epitope with which it immunoreacts. A monoclonal antibody may therefore contain an antibody molecule having a plurality of antibody combining sites, each immunospecific for a different epitope, e.g. a bispecific monoclonal antibody. Although historically a monoclonal antibody was produced by immortalization of a clonally pure immunoglobulin secreting cell line, a monoclonally pure population of antibody molecules can also be prepared by the methods of the present invention.

Laboratory methods for preparing monoclonal antibodies are well known in the art (see, for example, Harlow et al., 1988). Monoclonal antibodies (mAbs) may be prepared by immunizing purified Vanin-1 or pantetheine protein isolated from any of a variety of mammalian species into a mammal, e.g. a mouse, rat, rabbit, goat, camelides, human and the like mammal. The antibody-producing cells in the immunized mammal are isolated and fused with myeloma or heteromyeloma cells to produce hybrid cells (hybridoma). The hybridoma cells producing the monoclonal antibodies are utilized as a source of the desired monoclonal antibody. This standard method of hybridoma culture is described in Kohler and Milstein (1975).

While mAbs can be produced by hybridoma culture, the invention is not to be so limited. Also contemplated is the use of mAbs produced by an expressing nucleic acid cloned from a hybridoma of this invention. That is, the nucleic acid expressing the molecules secreted by a hybridoma of this invention can be transferred into another cell line to produce a transformant. The transformant is genotypically distinct from the original hybridoma but is also capable of producing antibody molecules of this invention, including immunologically active fragments of whole antibody molecules, corresponding to those secreted by the hybridoma. See, for example, U.S. Pat. No. 4,642,334 to Reading; PCT Publication No. WO 890099 to Robinson et al.; European Patent Publications No. 0239400 to Winter et al. and No. 0125023 to Cabilly et al.

Antibody generation techniques not involving immunisation are also contemplated such as for example using phage display technology to examine naive libraries (from non-immunised animals); see Barbas et al. (1992), and Waterhouse et al. (1993).

Aptamers may also be of interest. Aptamers are a class of molecule that represents an alternative to antibodies in term of molecular recognition. Aptamers are oligonucleotide or oligopeptide sequences with the capacity to recognize virtually any class of target molecules with high affinity and specificity. Oligonucleotidic aptamers may be isolated through Systematic Evolution of Ligands by EXponential enrichment (SELEX) of a random sequence library, as described in Tuerk C. and Gold L. (1990). The random sequence library is obtainable by combinatorial chemical synthesis of DNA. In this library, each member is a linear oligomer, eventually chemically modified, of a unique sequence. Possible modifications, uses and advantages of this class of molecules have been reviewed in Jayasena S. D. (1999). Peptide aptamers consists of a conformationally constrained antibody variable region displayed by a platform protein, such as E. coli Thioredoxin A that are selected from combinatorial libraries by two hybrid methods (Colas et al., 1996).

Alternatively, said Vanin-1 antagonist may be an indirect inhibitor of Vanin-1 activity that decreases its synthesis through inhibition of protein or gene expression. Inhibition of Vanin-1 expression may be achieved through blocking transcription of Vanin-1 gene and/or translation of Vanin-1 mRNA.

Antisense strategy may be used to interfere with Vanin-1 expression. This approach may for instance utilize antisense nucleic acids or ribozymes that block translation of a specific mRNA, either by masking this mRNA with an antisense nucleic acid or cleaving it with a ribozyme. For a general discussion of antisense technology, see, e.g., Antisense DNA and RNA, (Cold Spring Harbor Laboratory, D. Melton, ed., 1988).

Reversible short inhibition of Vanin-1 transcription may also be useful. Such inhibition can be achieved by use of siRNAs. RNA interference (RNAi) technology prevents the expression of genes by using small RNA molecules such as << small interfering RNAs” (siRNAs). This technology in turn takes advantage of the fact that RNAi is a natural biological mechanism for silencing genes in most cells of many living organisms, from plants to insects to mammals (Sharp, 2001). RNAi would prevent a gene from producing a functional protein by ensuring that the molecule intermediate, the messenger RNA copy of the gene is destroyed. siRNAs could be: used in a naked form and incorporated in a vector, as described below. A genetic construction containing a specific Vanin-1 RNAi capable of being cloned in at recombinant vector, such as a retrovirus, is shown in FIG. 8.

One can further make use of aptamers to specifically inhibit vanin-1 transcription.

An “antisense nucleic acid” or “antisense oligonucleotide” is a single stranded nucleic acid molecule, which, on hybridizing under cytoplasmic conditions with complementary bases in a RNA or DNA molecule, inhibits the latter's role. If the RNA is a messenger RNA transcript, the antisense nucleic acid is a countertranscript or mRNA-interfering complementary nucleic acid. As presently used, “antisense” broadly includes RNA-RNA interactions, RNA-DNA interactions, ribozymes, RNAi, aptamers and Rnase-H mediated arrest.

Ribozymes are RNA molecules possessing the ability to specifically cleave other single stranded RNA molecules in a manner somewhat analogous to DNA restriction endonucleases. Ribozymes were discovered from the observation that certain mRNAs have the ability to excise their own introns. By modifying the nucleotide sequence of these ribozymes, researchers have been able to engineer molecules that recognize specific nucleotide sequences in an RNA molecule and cleave it (Cech, 1989). Because they are sequence-specific, only mRNAs with particular sequences are inactivated.

Antisense nucleic acid molecules can be encoded by a recombinant gene for expression in a cell (e.g., U.S. Pat. No. 5,814,500; U.S. Pat. No. 5,811,234), or alternatively they can be prepared synthetically (e.g., U.S. Pat. No. 5,780,607).

As used herein, the term “oligonucleotide” refers to a nucleic acid, generally of at least 10, preferably at least 13, and more preferably at least 20 nucleotides, preferably no more than 100 nucleotides, that is hybridizable to a genomic DNA (gDNA) molecule, a complementary DNA (cDNA) molecule, or a messenger RNA (mRNA) molecule encoding a gene, mRNA, cDNA, or other nucleic acid of interest.

“Vanin-1 antisense” nucleic acids may be designed to specifically hybridize with a Vanin-1 encoding sequence, e.g. a sequence form the human Vanin-1 coding sequence shown in SEQ ID No 1.

A “sequence capable of specifically hybridizing with a nucleic acid sequence” is understood as meaning a sequence which hybridizes with the nucleic acid sequence to which it refers under the conditions of high stringency (Sambrook et al, 1989). These conditions are determined from the melting temperature Tm and the high ionic strength. Preferably, the most advantageous sequences are those which hybridize in the temperature range (Tm −5° C.) to (Tm −30° C.), and more preferably (Tm −5° C.) to (Tm −10° C.). A ionic strength of 6×SSC is more preferred. For instance, high stringency hybridization conditions correspond to the highest Tm, e.g., 50% formamide, 5× or 6×SCC. SCC is a 0.15 M NaCl, 0.015 M Na-citrate. Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the greater the value of Tm for hybrids of nucleic acids having those sequences. The relative stability (corresponding to higher Tm) of nucleic acid hybridizations decreases in the following order: RNA:RNA, DNA:RNA, DNA:DNA. For hybrids of greater than 100 nucleotides in length, equations for calculating Tm have been derived (see Sambrook et al., 1989). For hybridization with shorter nucleic acids, i.e., oligonucleotides, the position of mismatches becomes more important, and the length of the oligonucleotide determines its specificity (see Sambrook et al., 1989).

The antisense nucleic acid sequences according to the invention can be used as such, for example after injection into man or animal, to induce a protection or to treat an inflammatory and/or oxidative disorder. In particular, they can be injected in the form of naked DNA according to the technique described in application WO 90/11092. They can also be administered in complexed form, for example with DEAE-dextran (Pagano et al., 1967), with nuclear proteins (Kaneda et al., 1989), with lipids (Feigner et al., 1987), in the form of liposomes (Fraley et al., 1980), and the like.

Preferably, the nucleic acid sequences form part of a vector. The use of such a vector indeed makes it possible to improve the administration of the nucleic acid into the cells to be treated, and also to increase its stability in the said cells, which makes it possible to obtain a durable therapeutic effect. Furthermore, it is possible to introduce several nucleic acid sequences into the same vector, which also increases the efficacy of the treatment.

The terms “vector” means the vehicle by which a DNA or RNA sequence (e.g. a foreign gene) can be introduced into a host cell, so as to transform the host and promote expression (e.g. transcription and translation) of the introduced sequence. Vectors include plasmids, phages, viruses, etc.

The vector used in antisense strategy may be of diverse origin, as long as it is capable of transducing animal cells, and in particular human cells. In a preferred embodiment of the invention, a viral vector is used which can be chosen from adenoviruses, retroviruses, adeno-associated viruses (AAV), lentivirus, herpes virus, cytomegalovirus (CMV), vaccinia virus and the like. Vectors derived from adenoviruses, retroviruses or MVs, HIV-derived retroviral vectors, incorporating heterologous nucleic acid sequences have been described in the literature (Akli et al., (1993); Strafford-Perricaudet et al. (1990) EP 185 573, Levrero et al. (1991); Le Gal la Salle et al. (1993); Roemer et al (1992); Dobson et al. (1990); Chiocca et al. (1990); Miyanohara et al. (1992); WO 91/18088).

Such vectors generally comprise a promoter sequence, signals for initiation and termination of transcription. Their insertion into the host cell may be transient or stable. These various control signals are selected according to the host cell and may be inserted into vectors which self-replicate in the selected host cell, or into vectors which integrate the genome of said host.

The present invention therefore also relates to any recombinant virus comprising, inserted into its genome, a Vanin-1 antisense sequence.

Advantageously, the recombinant virus according to the invention is a defective virus. The term “defective virus” designates a virus incapable of replicating in the target cell. Generally, the genome of the defective viruses used within the framework of the present invention is therefore devoid of at least the sequences necessary for the replication of the said virus in the infected cell. These regions can either be removed (completely or partially), or rendered non-functional, or substituted by other sequences and especially by the Vanin-1 antisense nucleic acid of the invention. Preferably, the defective virus nevertheless conserves the sequences of: its genome which are necessary for the encapsulation of the viral particles.

It is particularly advantageous to use the nucleic acid antisense sequences of the invention in a form incorporated in an adenovirus, an MV or a defective recombinant retrovirus.

As regards adenoviruses, various serotypes exist whose structure and properties vary somewhat, but which are not pathogenic for man, and especially non-immunosuppressed individuals. Moreover, these viruses do not integrate into the genome of the cells which they infect, and can incorporate large fragments of exogenous DNA Among the various serotypes, the use of the AD5/F35 chimeric adenovirus vector (Yotnda et al., 2001) is preferred within the framework of the present invention. In the case of the Ad5 adenoviruses, the sequences necessary for the replication are the E1A and E1B regions.

The defective recombinant viruses of the invention can be prepared by homologous recombination between a defective virus and a plasmid carrying, inter alia, the Vanin-1 antisense nucleic acid sequence (Levrero et al., 1991; Graham, 1984). The homologous recombination is produced after co-transfection of the said viruses and plasmid into an appropriate cell line. The cell line used should preferably (i) be transformable by the said elements, and (ii), contain sequences capable of complementing the part of the genome of the defective virus, preferably in integrated form so as to avoid the risks of recombination. As example of a line which can be used for the preparation of defective recombinant adenoviruses, there may be mentioned the human embryonic kidney line 293 (Graham et al., 1977) which contains especially, integrated into its genome, the left part of the genome of an Ad5 adenovirus (12%). As example of a line which can be used for the preparation of defective recombinant retroviruses, there may be mentioned the CRIP line (Danos et al., 1988). Alternative vectors, such as shuttle vectors, can also be used that permit the cloning of the desired gene in the vector backbone.

Then the viruses that have multiplied are recovered and purified according to conventional molecular biology techniques.

Vectors insertion into the host cell may be achieved by transfection or infection.

Antisense oligonucleotides can also be used to provide a transient Vanin-1 inhibition. For that purpose antisense oligonucleotides, that are not part of a viral vector, can be administered to the cell by any means as described below.

The term “transfection” means the introduction of a foreign nucleic acid into a cell. The term “transformation” means the: introduction of a “foreign” (i.e. extrinsic or extracellular) gene, DNA or RNA sequence to a host cell, so that the host cell will express the introduced gene or sequence to produce a desired substance, typically an antisense sequence.

The term “host cell” means any cell of any organism that is selected, modified, transformed, grown, or used or manipulated in any way, for the production of a substance by the cell, for example the expression by the cell of a gene, a DNA or RNA sequence, a protein or an enzyme.

Targeted gene delivery is described in International Pat. Publication WO 95/28494, published October 1995.

Alternatively, the antisense nucleic acid sequence, which may be part or not of a vector, can be introduced in vivo by lipofection. For the past decade, there has been increasing use of liposomes for encapsulation and transfection of nucleic acids in vitro. Information regarding liposome is provided in the “pharmaceutical composition” section of the present application as well. Synthetic cationic lipids designed to limit the difficulties and dangers encountered with liposome mediated transfection can be used to prepare liposomes for in vivo transfection of a nucleic acid sequence (Felgner et al., 1987). The use of cationic lipids may promote encapsulation of negatively charged nucleic acids, and also promote fusion with negatively charged cell membranes (Felgner et al., 1989). The use of lipofection to introduce exogenous genes into the specific organs in vivo has certain practical advantages. Molecular targeting of liposomes to specific cells represents one area of benefit. It is clear that directing transfection to particular cell types would be particularly advantageous in a tissue with cellular heterogeneity, such as pancreas, liver, kidney, and the brain. Lipids may be chemically coupled to other molecules for the purpose of targeting. Targeted peptides, e.g. hormones or neurotransmitters, and proteins such as antibodies, or non-peptide molecules could be coupled to liposomes chemically.

It is also possible to introduce in vivo the antisense nucleic acid sequence, which may be part or not of a vector, as a naked DNA plasmid. Naked DNA vectors can be introduced into the desired host cells by methods known in the art, e.g., transfection, electroporation, microinjection, transduction, cell fusion, DEAE dextran, calcium phosphate precipitation, use of a gene gun, or use of a DNA vector transporter (see, e.g., Wilson et al., 1992; Wu et al., 1988).

### Screening Methods

Based on the inventors' results showing that Vanin-1 is involved in the deregulation of GSH homeostasis in face of oxidative stress, thus preserving tissue integrity and function upon inflammatory treatments, a method for identifying compounds useful for the treatment of an inflammatory and/or an oxidative disorder is proposed.

Accordingly, the present invention relates to a method for identifying a compound useful for the treatment of an inflammatory and/or an oxidative disorder, comprising the steps consisting of determining the capacity of a test compound having Vanin-1 antagonist activity to prevent or inhibit an induced inflammation and/or oxidative disorder or state.

In the context of the invention, the test compound having Vanin-1 antagonist activity may be an already known Vanin-1 antagonist, or a compound the Vanin-1 antagonist activity is not known but that can be assayed before or after implementation of the method of screening of the invention. In particular Vanin-1 inhibitory activity may be readily assessed by the one skilled in the art according to a method as described above.

As used herein, an “induced inflammation and/or oxidative condition” denotes a condition as can be observed in a cell, a tissue or an animal, following exposure to an oxidative stress and/or a pro-inflammatory signal. Said condition may be elicited for instance upon acute or chronic administration of a chemical, such as H₂O₂, or a non-steroidal anti-inflammatory drug, or trinitrobenzenesulphonic acid (TNBS), or of pro-inflammatory cytokines such as interleukine-1β (IL-1β) or Tumor Necrosis Factor α (TNFα), or irradiation such as gamma irradiation. TNBS induced-colitis in mammal constitutes a model for chronic cholangitis, Crohn's disease or ulcerative colitis (Orth et al., 2000; Neurath et al., 2000). Alternatively, said condition may be generated following genetic manipulation of a cell or an animal.

Preferably, said inflammatory and/or oxidative disorder is a gastrointestinal inflammatory disorder, still preferably an NSAID-induced ulcer, Crohn's disease or ulcerative colitis, or a Scistosomiaisis, i.e. an inflammatory disorder associated with an infection with Schistosoma mansoni parasite.

The anti-inflammatory and/or anti-oxidative activity of the test compound may be assayed in any suitable ex vivo cellular or tissue-based model assay, or in vivo in animal models of inflammatory and/or an oxidative disorder.

According to a first embodiment, the invention relates to a method for identifying a compound useful for the treatment of an inflammatory and/or an oxidative disorder, comprising the steps consisting of:

a) administering a mammal (i) with an inducer of an inflammatory and/or oxidative disorder, in a amount suitable for inducing inflammatory and/or an oxidative disorder in said mammal and (ii) a test compound; and

b) determining the capacity of the test compound to prevent or inhibit the inflammatory and/or oxidative disorder in said mammal, wherein a reduced inflammation and/or oxidative disorder, or a non-appearance thereof, is indicative of a compound useful for the treatment of an inflammatory and/or oxidative disorder.

The test compound may be administered prior to the inducer of an inflammatory and/or oxidative disorder, and the inflammatory and/or oxidative disorder of the mammal may be assessed before and after each administration, and compared with the inflammatory and/or oxidative disorder of a control mammal to which a control vehicle has been administered in lieu of the test compound. Accordingly, a reduction of inflammatory and/or oxidative disorder, or non-appearance thereof, with comparison to the control mammal is indicative of a test compound having Vanin-1 antagonist activity useful for the treatment of an inflammatory and/or oxidative disorder.

Alternatively, the test compound may be administered after the inducer of an inflammatory and/or oxidative disorder, and the inflammatory and/or oxidative disorder of the mammal may be assessed before and after each administration, and compared with the inflammatory and/or oxidative disorder of a control mammal to which a control vehicle has been administered in lieu of the test compound. A reduced inflammatory and/or oxidative disorder with comparison to the control mammal is indicative of a test compound having Vanin-1 antagonist activity useful for the treatment of an inflammatory and/or oxidative disorder.

According to a second embodiment, a tissue-based ex vivo assay may be used for detecting Vanin-1 antagonists with anti-inflammatory and/or anti-oxidative activity. According to this assay, parts of gut, preferably parts of ileon and/or colon, are isolated from normal intestinal transit and ex-vivo manipulated while retaining vascularisation on tissues of anesthetized mammal.

Inventors have developed a mouse model in which mice are anesthetised, and ileon or colon is externalised. Every 2 cm, transit is interrupted by ligation without disconnecting from the vascularisation. Every segment can be manipulated independently by simple injection in the intestinal lumen of any compound (for instance a drug or a Vanin-1 antagonist such as described above) and followed for a couple of hours. At the end of the experiment, each segment is cut and processed for immunohistochemistry, biochemical or molecular analysis.

This protocol allows an easy access and manipulation of the content of gut for at least two days. It is easily amenable to pharmacological manipulations since one can inject locally precise quantities of compounds, such as Vanin-1 antagonists, or chemicals such as cysteamine or cystamine, or inflammation-inducing drugs, thus avoiding in vivo toxicity and permitting a precise adjustment of delivered quantities.

Accordingly, the invention provides an ex vivo method for identifying a compound useful for the treatment of an inflammatory and/or an oxidative disorder, comprising the steps consisting of:

a) contacting a gut sample from a mammal with an inducer of an inflammatory disorder and/or oxidative disorder, and a test compound

b) determining the capacity of the test compound to prevent or inhibit the inflammation and/or oxidative disorder induced in gut, wherein a reduced inflammation and/or oxidative disorder, or a non-appearance thereof, is indicative of a compound useful for the treatment of an inflammatory and/or oxidative disorder.

The test compound may be administered prior to the inducer of an inflammatory and/or oxidative disorder, and the inflammatory and/or oxidative disorder of the gut sample may be assessed before and after each administration, and compared with the inflammatory and/or oxidative disorder of a control gut sample to which a control vehicle has been administered in lieu of the test compound. Accordingly, a reduction of inflammatory and/or oxidative disorder, or non-appearance thereof, with comparison to the control gut sample is indicative of a test compound having Vanin-1 antagonist activity useful for the treatment of an inflammatory and/or oxidative disorder.

Alternatively, the test compound may be administered after the inducer of an inflammatory and/or oxidative disorder, and the inflammatory and/or oxidative disorder of the gut sample may be assessed before and after each administration, and compared with the inflammatory and/or oxidative disorder of a control gut sample to which a control vehicle has been administered in lieu of the test compound. A reduced inflammatory and/or oxidative disorder with comparison to the control mammal is indicative of a test compound having Vanin-1 antagonist activity useful for the treatment of an inflammatory and/or oxidative disorder.

According to these two first embodiments, the term “inducer of an inflammatory and/or oxidative disorder” is intended for NSAID, TNBS, Schistosoma mansoni, H₂O₂, TBHQ (t-butyl-hydroquinone), irradiation such as gamma irradiation or any other agent with pro-inflammatory or pro-oxidative activity well known in the art. Said inducer may be: administered or applied, where appropriate, acutely, continuously, i.e. by infusion, or chronically to the mammal or gut sample for a time sufficient to allow development of an inflammatory and/or oxidative disorder. However, said inducer of an inflammatory and/or oxidative disorder may be administered in any form and according to dosage suitable for inducing an inflammation and/or an oxidative disorder. Such a dosage may be comprised between 10 and 100 mg/kg sc. For example, indomethacin is administered at a dose of 2×25 mg/kg sc.

A test compound may be administered according to dosage established in the art whenever specific blockade of the Vanin-1 activity or expression is required.

Preferably, in the above first and second embodiments, said mammal is a non-human mammal, still preferably a mouse or a rat.

According to the above animal or tissue based assays, the capacity of the test compound to prevent or inhibit an induced inflammation and/or oxidative condition may be readily determined for instance by.

1) assaying secretion of metabolites that are the hallmark of an inflammatory and/or oxidative disorder, such as pro-inflammatory lipids (for instance prostaglandins, leucotrienes (leucotrienes B4, cysteinyl leucotrienes), platelet-activating factor) and/or anti-inflammatory lipids (for instance cyclopentones prostaglandins, lipoxins) (Smith, 2000; Funk 2000). These lipids may be assayed either by commercially available kits or by HPLC analysis following injection of radiolabelled precursor (arachidonic acid).

2) scoring the level of epithelial cell damage by immunohistochemistry or biochemical techniques. For instance, histological scoring for infiltration, ulceration, necrosis mucus secretion, proliferation/apoptosis, length of villi can be carried out as described in Desreumaux et al. (2001).

3) quantifying epithelial cell signalling by any suitable immunochemical technique, or a combination thereof, preferably by performing a Western blot analysis on tissue extracts using a variety of antibodies directed at key components of signalling pathway, said components being kinases, phosphatases, transcription factors such as NF-kB, p38MAPK, JNK, AP-1 (Desreumaux, 2001).

For instance, the capacity of the test compound to prevent or inhibit an induced inflammation and/or oxidative disorder may be determined by quantifying inflammatory factors mRNAs and/or protein expression, in the presence or not of the test compound. Such factors may be selected from the group consisting of INOS (inducible nitric oxide synthetase; Lyons, 1992), COX-1 (cyclooxigenase-1; Ballou, 2000), COX-2 (Scoff, 2001), MIP-2 (macrophage inflammatory protein-2; Ohtsuka, 2001), Interleukin-1, Interleukin-2, TNF-alpha (Tumor Necrosis Factor). A decreased or absence of inflammatory factors following administration of an inducer of an inflammatory and/or oxidative disorder is indicative of a test compound with anti-inflammatory or anti-oxidative activity.

Other examples include assay of tissue myeloperoxidase activity, as carried out in the following examples, or detection of nitrosylated proteins, or apoptosis (TUNEL method for instance). Useful methods for identifying endogenously nitrosylated proteins are the “biotin switch” or the immunoprecipitation methods described by Mannick and Schonhoff (2002).

According to a third embodiment, an in vitro cellular model of inflammation and/oxidative stress may be used; for detecting Vanin-1 antagonists with anti-inflammatory and/or anti-oxidative activity.

The system described in Kerneis et al. (1997) may be adapted to detect Vanin-1 antagonists with anti-inflammatory and/or anti-oxidative activity. In this adapted system, epithelial cells are cultured on the bottom of the Transwell™ system (Costar Corporation) described in U.S. Pat. No. 5,026,649, in which cells or tissue samples are separated from a nutrient medium by a permeable membrane. Once confluence is reached, labelled leucocytes are added to the top chamber and various stresses can; be added in the bottom compartment of the Transwell system. Transmigration of leucocytes across the epithelial layer could be quantified as a measure of chemottractant secretion (MIP-2) induced by the epithelial stress.

Reference can be also made for instance to the international patent application WO 00/42429 that describes an in vitro model for gastrointestinal inflammation. This system is also derived from the Transwell™ system (Costar Corporation). Such a system makes it possible to mimic normal and disease states, and thus to screen for molecule having anti-inflammatory activity.

Accordingly, the invention provides an in vitro method for identifying a compound useful for the treatment of an inflammatory and/or an oxidative disorder, comprising the steps consisting of:

a) contacting a cell with an inducer of an inflammatory and/or oxidative state, and a test compound

b) determining the capacity of the test compound to prevent or inhibit the inflammation and/or oxidative state induced in said cell, wherein a reduced inflammation and/or oxidative state, or a non-appearance thereof, is indicative of a compound useful for the treatment of an inflammatory and/or oxidative disorder.

The test compound may be contacted with the cell prior to the inducer of an inflammatory and/or oxidative state, and the inflammatory and/or oxidative state of the cell may be assessed before and after each contacting, and compared with the inflammatory and/or oxidative state of a control cell that has been contacted with a control vehicle in lieu of the test compound. Accordingly, a reduction of inflammatory and/or oxidative state, or non-appearance thereof, with comparison to the control cell is indicative of a test compound having Vanin-1 antagonist activity useful for the treatment of an inflammatory and/or oxidative disorder.

Alternatively, the test compound may be contacted with the cell after the inducer of an inflammatory and/or oxidative state, and the inflammatory and/or oxidative condition of the cell may be assessed before and after each contacting, and compared with the inflammatory and/or oxidative state of a control cell that has been contacted with a control vehicle in lieu of the test compound. A reduced inflammatory and/or oxidative state with comparison to the control cell is indicative of a test compound having Vanin-1 antagonist activity useful for the treatment of an inflammatory and/or oxidative disorder.

Preferably, said inducer of an inflammatory and/or oxidative state may be selected from the group consisting of IL-1β, IFNα, TNFα, H₂O₂, TBHQ (ter-butyl-hydroquinone), irradiation such as gamma, irradiation and paraquat (1,1′-diméthyl-4,4′-bipyridinium; the mechanism of paraquat toxicity in lung has been described by Smith (1987)).

Advantageously, said cell is an epithelial cell, preferably from the gastrointestinal tract. Epithelial cells suitable for use can be obtained from any in vivo source, including but not limited to epithelial cells derived from the murine or human gastrointestinal tract. CaCo-2 cells, or derivatives thereof, are most preferred, and are available commercially from culture collection banks such as the ATCC and ECACC Procedures for harvesting and culturing epithelial cells are well-known in the art.

Furthermore, said cell may be a cell from an organism (for instance bacteria, yeast, fungus) which does not have nor express Vanin-1 gene and which has been transfected in order to express Vanin-1 protein, preferably human VNN-1 or mouse Vanin-1 protein. Procedures to modify genetically such a cell to make it express a protein are well-known in the art.

The inflammatory and/or oxidative state of a cell may be determined by assaying key components of the inflammatory and/or oxidative signalling pathway. As described above, INOS, COX-1, COX-2, and/or MIP-2 mRNAs, SOD, catalase, glutathion (GSH), γ-GCS, GPX or GST activities may be quantified to determine the oxidative state of a cell.

### Therapeutic Methods

The inventors have shown that blocking Vanin-1 function makes it possible to prevent or inhibit development of an inflammatory condition in various models of inflammation, in particular NSAID-induced inflammation and Crohn's disease.

Therefore, a subject of the invention is a method for the treatment of an inflammatory and/or oxidative disorder that comprises administrating a patient in need thereof with a therapeutically effective amount of a Vanin-1 antagonist, in a pharmaceutically acceptable carrier.

Accordingly, the invention also relates to the use of a Vanin-1 antagonist for the manufacture of a medicament intended for the treatment of an inflammatory disorder and/or oxidative condition.

The Vanin-1 antagonists of the invention are useful for the treatment of inflammatory disorder and/or an oxidative condition, preferably for the treatment of a gastrointestinal inflammatory disorder.

Preferably, the Vanin-1 antagonist has anti-inflammatory or anti-oxidative activity, in vitro or in vivo. Such an activity may be readily assessed by the one skilled in the art using one of the methods described above.

In the context of the present invention, the term “pharmaceutically” or “pharmaceutically acceptable” refer to molecular entities and compositions that do not produce an adverse, allergic or other untoward reaction when administered to an animal, or a human, as appropriate. The Vanin-1 antagonist may thus be mixed with a pharmaceutically acceptable carrier compatible with the Vanin-1 antagonist and in amounts suitable for use in the therapeutic methods described herein.

As used herein, “Pharmaceutically acceptable carrier” includes any and all solvents, dispersion composition, coatings, antibacterial and antifungal agents, isotonic and absorption delaying agents and the like. Suitable carrier are, for example, water, saline, dextrose, glycerol, ethanol or the like and combinations thereof. These compositions and agents do not produce an adverse, allergic or other untoward reaction when administered to an animal, or a human, as appropriate. They are selected on the basis of the mode and route of administration, and standard pharmaceutical practice. The use of such composition and agents for pharmaceutical active substances is well known in the art. Except insofar as any conventional composition or agent is incompatible with the active ingredient, its use in the therapeutic compositions is contemplated. Supplementary active ingredients can also be incorporated into the compositions. In addition, if desired, the composition can contain minor amounts of auxiliary substances such as wetting or emulsifying agents, pH buffering agents and the like which enhance the effectiveness of the active ingredient.

The term “patient”, or “patient in need thereof”, is intended for a human or non-human mammal affected or likely to be affected with an inflammatory and/or oxidative disorder, such a human or non-human mammal treated with a NSAID as described above.

In the above therapeutic methods or applications, the Vanin-1 antagonist, may be administered orally, parenterally (including subcutaneous injections, intravenous, intramuscular, intrasternal injection or infusion techniques), by inhalation spray, or rectally, in dosage unit formulations containing conventional non-toxic pharmaceutically-acceptable carriers, adjuvants and vehicles.

Such pharmaceutical compositions containing a Vanin-1 antagonist alone or in combination with another Vanin-1 antagonist or another active ingredient may thus be in the form of orally-administrable suspensions or tablets; nasal sprays; sterile injectable preparations, for example, as sterile injectable aqueous or oleagenous suspensions or suppositories.

When administered orally as a suspension, these compositions are prepared according to techniques well-known in the art of pharmaceutical formulation and may contain microcrystalline cellulose for imparting bulk, alginic acid or sodium alginate as a suspending agent, methylcellulose as a viscosity enhancer, and sweeteners/flavoring agents known in the art. As immediate release tablets, these compositions may contain microcrystalline cellulose, dicalcium phosphate, starch, magnesium stearate and lactose and/or other excipients, binders, extenders, disintegrants, diluents and lubricants known in the art.

When administered by nasal aerosol or inhalation, these compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

The injectable solutions or suspensions may be formulated according to known art, using suitable non-toxic, parenterally-acceptable diluents or solvents, such as mannitol, 1,3-butanediol, water, Ringer's solution or isotonic sodium chloride solution, or suitable dispersing or wetting and suspending agents, such as sterile, bland, fixed oils, including synthetic mono- or diglycerides, and fatty acids, including oleic acid.

When rectally administered in the form of suppositories, these compositions may be prepared by mixing the drug with a suitable non-irritating excipient, such as cocoa butter, synthetic glyceride esters or polyethylene glycols, which are solid at ordinary temperatures, but liquidify and/or dissolve in the rectal cavity to release the drug.

A Vanin-1 antagonist may be administered in any of the foregoing compositions and according to dosage regimens, established in the art whenever specific blockade of the Vanin-1 activity or expression is required.

The daily dosage of the products may be varied over a wide range from 0.01 to 1,000 mg per adult human per day. For oral administration, the compositions are preferably provided in the form of tablets containing 0.01, 0.05, 0.1, 0.5, 1.0, 2.5, 5.0, 10.0, 15.0, 25.0, 50.0, 100, 250 and 500 milligrams of the active ingredient for the symptomatic adjustment of the dosage to the patient to be treated. A medicament typically contains from about 0.01 mg to about 500 mg of the active ingredient, preferably, from about 1 mg to about 100 mg of active ingredient. An effective amount of the drug is ordinarily supplied at a dosage level of from about 0.0002 mg/kg to about 20 mg/kg of body weight per day. Preferably, the range is from about 0.001 to 10 mg/kg of body weight per day, and especially from about 0.001 mg/kg to 7 mg/kg of body weight per day. The compounds may be administered on a regimen of 1 to 4 times per day. It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

The invention will be further illustrated by the following figures and examples.

### LEGEND OF THE FIGURES

FIG. 1 is a graph showing prolonged survival of S. mansoni-infected Vanin-1^{-/-} mice. Survival curves of Vanin-1^{-/-} (square) and WT (O) mice infected with a lethal dose of S. mansoni cercariae. Dashed lines delimitate the time period (8 to 12 weeks post infection (p.i.)) during which hemoglobin amount in feces was daily assayed.

FIG. 2 is a graph showing the daily quantification of the hemoglobin amount in S. mansoni infected Vanin-1^{-/-} and WT mice feces. The shaded scale indicates Hb amount, namely <7.5, 7.5-24, 24-60 and >60 ng Hb/mg excrement. (+), dead mice.

FIG. 3 shows reduced expression of inflammatory factors in Vanin-1^{-/-} mice. (A) is a representative experiment of semi-quantitative RT-PCR analysis of MIP-2, iNOS, COX-2, and COX-1 mRNAs in the jejunum of untreated or indomethacin-treated Vanin-1^{-/-} and WT mice. Cystamine was administered when indicated. Amplification of HPRT mRNA transcript was used as internal control. (B) is a graph showing lower intestinal MPO activity in S. mansoni infected and indomethacin-treated Vanin-1^{-/-} mice. (hachured square) Vanin-1^{-/-}; (open square), WT. Values are means ±SD. (*), values from Vanin-1^{-/-} and WT groups are significantly different, P<0.05, Student's t test.

FIG. 4 represents γ-GCS increased (A) and GSH stores in liver of Vanin-1^{-/-} mice following S. mansoni infection and indomethacin treatment. Cystamine was administered when indicated. (hachured square) Vanin-1^{-/-}; (open square), WT. Values are means ±SD. (*), values from Vanin-1^{-/-} and WT groups are significantly different, P<0.05, Student's t test.

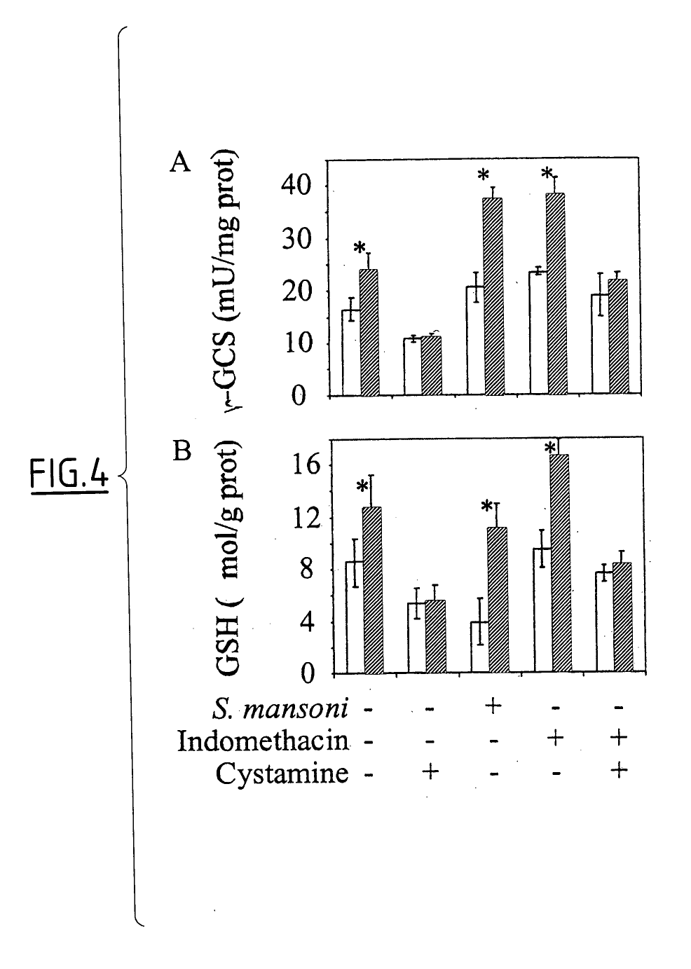

FIG. 5 represents thymic reconstitution (A, B, C) and inflammatory response (D) following γ-irradiation (6 gy) of WT and Vanin-1^{-/-} mice. (A) represents total thymic cell number of (i) WT mice, whose cells have been treated with anti-Vanin-1 mAbs subclones (407.6.3 and 407.7.4), or 734 antibody or PBS as in Aurrand-Lions et al. (1996) and (ii) Vanin-1^{-/-} mice whose cells have boon treated with anti-Vanin-1 407.6.3 mAb or PBS; (B) represents the number of CFDA-SE+ cells in thymus 3 days; following irradiation. (C) represents the total cell number in thymus 8 days post-irradiation (D) is a representative experiment of semi-quantitative RT-PCR analysis of IL1, IL6, MIP-2, INOS, and KGF mRNAs in thymus.

FIG. 6 shows decreased selenium-independent glutathione peroxidase (GPx-Se−) activities in liver and thymus of Vanin-1^{-/-} (hachured square) mice as compared to WT (open square).

FIG. 7 is a graph showing prolonged survival of Vanin-1^{-/-} mice treated with TNBS.

FIG. 8 represents a recombinant vector containing a specific Vanin-1 RNAi. The cloning vector used is pSUPER-GFP (Brummelkamp, 2002) and the retroviral vector is LZRS (Heemskerk, 1999).

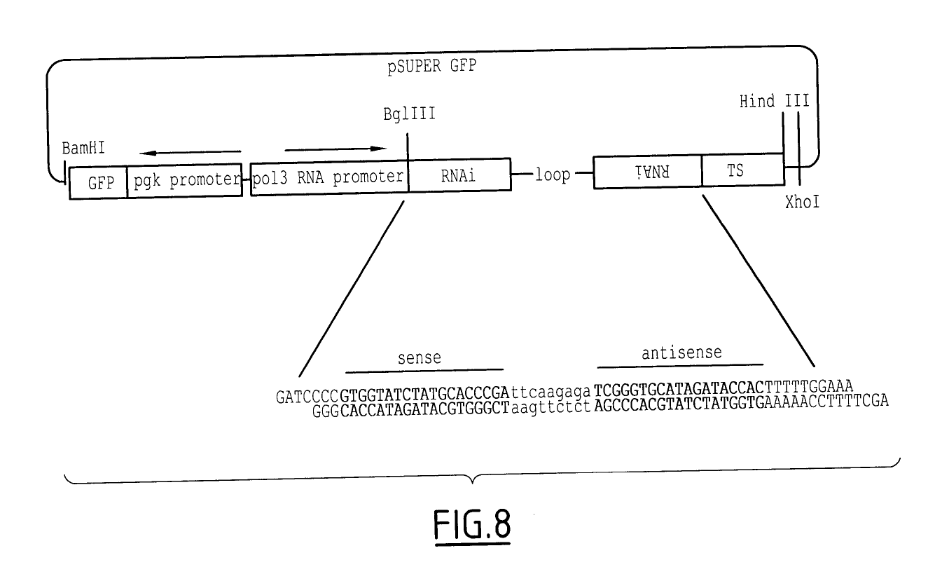

### EXAMPLES

### Example 1

### Material and Methods

1.a) Animal Model

Vanin-1^{-/-} mice (from the Centre d'Immunologie de Marseille Luminy—France; described in Pitari, 2000) backcrossed on a BALB/c background (9 generations) were kept in a specific pathogen-free mouse facility and handled according to the rules of <<(Décret no 87-848 du 19 Oct. 1987. Paris>>. Mice had free access to water and to complete rodent diet (UAR, France).

1.b) Infection with S. mansoni.

2 cohorts of Vanin-1^{-/-} (n=10, each) and WT (n=10) mice were percutaneously infected with 150 cercariae of S. mansoni (Puerto Rican strain). A first cohort was used for survival studies. Mice of the second cohort were sacrificed 8 weeks post-infection (p.i.), for determination of pathophysiological parameters.

1.c) Survival Studies.

Survival of mice has been followed daily after S. mansoni infection. During the acute phase of the disease (8 to 12 weeks p.i.) (Wynn, 1998), the severity of intestinal injury was monitored by quantifying the presence of hemoglobin in feces. For this purpose, droppings were collected daily and resuspended in a 1:5 w/v ratio of distilled water. Samples were diluted 10 times and the hemoglobin concentration determined using Hemastix Reagent Strips (Bayer). Results were expressed in ng of hemoglobin per mg of excrement.

1.d) Drug Administration.

Indomethacin (Sigma) was administered at a dose of 25 mg/kg in 5% sodium carbonate to Vanin-1^{-/-} (n=5) and WT (n=5) mice in 2 SC injections at 12 h-interval. Mice were sacrificed 20 hrs after the first injection. The treatment induced non-ulcerative intestinal inflammation as previously described (Beck, 2000). Cystamine was given by gavage 3 times on day 1 and once on day 2 at the dose of 120 mg/kg. The dose was adjusted so as to not induce intestinal inflammation. Mice were sacrificed 5 hours after the last gavage. To evaluate the impact of cystamine on indomethacin treatment, Vanin-1^{-/-} (n=5) and WT (n=5) mice received cystamine preceding indomethacin injection. In a complement study, indomethacin-treated WT mice (n=5) were supplemented with N-acetylcysteine (NAC) to determine the relationship between GSH depletion and injury. The antioxidant was given by gavage (200 μl of a 150 mg/ml, pH 7 solution) three times a day, starting 12 hrs prior indomethacin treatment.

1.e) Histopathological Analysis.

After sacrifice, jejunum of the indomethacin-treated mice were removed, formalin-fixed and paraffin-embedded. They were cut to 4-μm thickness and stained with hematoxylin-phloxine-saffron. Immunohistochemistry was performed on jejunum cryosections using the anti-Vanin-1 mAb H202-407 modified from Aurrand-Lions et al. (Aurrand-Lions, 1996). The primary mAb was revealed using a mouse-adsorbed goat anti-rat Ig-HRP (Southern Biotechnology associates, Birmingham, Ala.) using cyanin-3-tyramide as substrate (PerkinElmer Life Sciences, Boston, Mass.).

Thymuses from irradiated or non-irradiated Vanin-1^{-/-} and WT mice were embedded in Tissue-Tek OCT compound (Sakura) and frozen. Aceton fixed 8 μm-thick cryosections were incubated with the 407 mAb (Aurrand-Lions, 1996) and washed three times prior to be incubated with a mouse-adsorbed goat anti-rat Ig-HRP (Southern Biotechnology associates, Birmingham, Ala.) as a secondary reagent (Van Vliet et al. 1984). Sections were then washed three times in a buffer saturated with 10% normal rat serum (TEBU CALTAG Laboratories) and incubated with various FITC coupled antibodies (29, 95, CD31, 735 or B-220, anti IgM, anti IgD as isotypic negative controls) diluted in 5% normal rat serum blocking buffer. After 3 washes, the 407 mAb staining was revealed using a cyanin-3-tyramide substrate (Perkin Elmer Life Sciences, Boston, Mass.). Microscopy analysis was performed using an Axiovert microscope coupled to an Axiocam MRC camera (ZEISS) and a confocal Leica microscope.

1.f) Myeloperoxidase (MPO) Activity.

3-cm intestinal samples taken in the most injured part, i.e. the colon of S. mansoni-infected mice and the jejunum of indomethacin-treated mice. Samples were homogenized in 0.5% hexadecyltrimethyl-ammonium bromide. MPO activity was determined as described previously (Liaudet, 2000).

1.g) RNA Analysis.

Total cellular RNA was isolated from jejunum samples of indomethacin-treated mice, by guanidinium thiocyanate phenol-chloroform extraction. INOS (inducible nitric oxide synthetase) (Lyons, 1992), COX-1 (cyclooxigenase-1) (Ballou, 2000), COX-2 (Scott, 2001) and MIP-2 (macrophage inflammatory protein-2) (Ohtsuka, 2001) mRNAs were measured by semi-quantitative RT-PCR, using hypoxanthine-guanine phosphoribosyltransferase (HPRT) mRNA (Konecki, 1982) as a quantitative reference.

1.h) Determination of γ-GCS Activity and GSH Levels in Liver and Intestine.

γ-GCS activity was determined by the method described by (Seelig, 1984). GSH levels were determined according to Tietze et al., (1969).

1.j) Statistical Analysis.

Data are expressed as mean ±SD (standard deviation). Values from experimental and control groups were compared using the Student's t test. Probability values P<0.05 were considered statistically significant.

1.k) Thymus Reconstitution Experiments.

For irradiation experiments, age and weight of Vanin-1^{-/-} mice matched wild-type. Mice were submitted to sublethal γ-irradiation (6 gy).

On day 3 (short term) and 8 (long term) after irradiation, thymuses were removed for cell numbering by trypan blue exclusion. Thymocyte suspensions were adjusted to 20.10⁶ cells/ml. Cytofluorometric analysis was performed on a FACS analyzer (Becton Dickinson) using combinations of CD3-PE, CD4-biotin, CD8α-FITC, CD25-FITC and CD117-biotin antibodies (Pharmingen) for long term experiments and combinations of CD3-PE, CD4-biotin, CD8α-PE, CD25-PE, CD11c-biot, TCRαβ-biot, TCRγδ-biot, Mac-1-PE and CD117-biotin antibodies (Pharmingen) for short term experiments.

For short term thymus reconstitution experiments (3 days), bone marrow cells were labeled during 7 min at +37° C. with 10 μM CFDA-SE (Molecular Probes) at a concentration of 10⁷ cells per ml in PBS (Invitrogen). 30.10⁶ labeled cells were injected intravenously into each irradiated receiver.

1.l) TNBS Administration

Mice are fasted for 1 day, anesthetized, and TNBS (trinitrobenzene sulfonic acid—Sigma) is administered with a catheter inserted into the colon such as the tip was 4 cm proximal to the anus. To induce colitis a dose of 3 mg/kg in 50% ethanol (100 ul volume) is injected to Vanin-1^{-/-} (n=17) and WT (n=27) mice. Animals were monitored to loss of body weight and survival

### EXAMPLE 2

### Results

2.a) Vanin-1^{-/-} Mice Survived Longer Following S. mansoni Infection

In the S. mansoni infection model, parasite eggs target both liver and intestine, leading to periportal fibrosis and intestinal lesions, respectively. On the intestinal site, the main pathological syndrome is diffuse hemorrhages (Cheever, 1985).

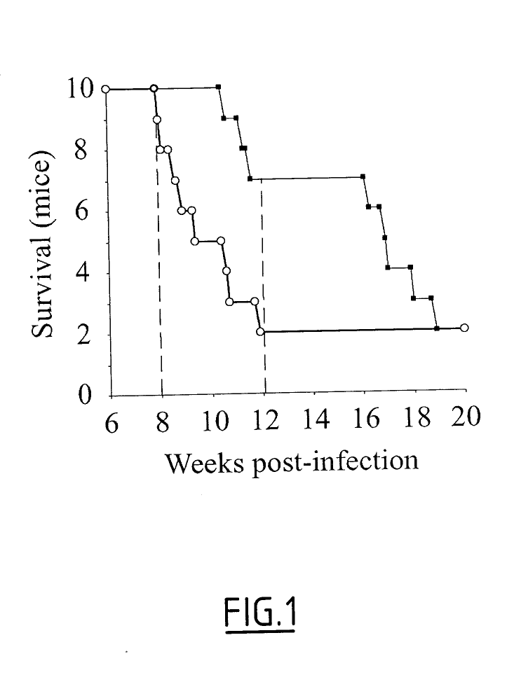

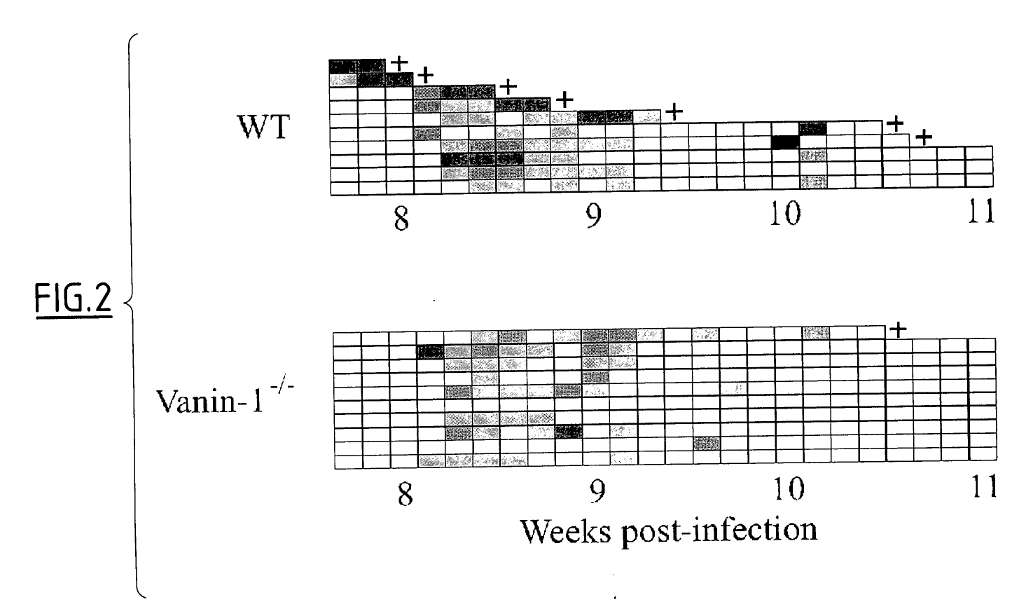

The 8-12 week post-infection period is the acute phase of S. mansoni-induced inflammation. This period was critical for survival of WT mice, as 8 out of 10 WT mice died. Mortality was delayed in the Vanin-1^{-/-} group and only 3 out of 10 mice died during acute-phase period. Then, survival in the mutant group was stable for 4 weeks until deaths resumed at 16 weeks p.i. (FIG. 1). In both groups 2 mice were still alive at the date of sacrifice (20 weeks p.i.). Among the 8 S. mansoni infected WT mice which died during the 8-12 week p.i. period, 5 showed severe intestinal bleeding, with a very high hemoglobin (Hb) level in the feces (FIG. 2). At autopsy, their cecum and colon were swollen and infiltrated with blood. Such an appearance was never seen for Vanin-1^{-/-} mice, which had less abundant and more transient intestinal bleeding than WT mice. Also, equivalent numbers of eggs were found in intestinal wall of both WT and Vanin-1^{-/-} mice, suggesting that expression of Vanin-1 had no effect on the parasite burden. Egg deposition led to widespread diffuse intestinal lesions (Cheever, 1985) leading to hemorrhages. Further, the intestinal injury was dramatically reduced in the absence of Vanin-1.

Vanin-1 deficient mice that survived to intestinal injury later died from liver disease, since disease progression in the liver proceeded similarly in Vanin-1^{-/-} and WT mice. This discrepancy between liver and intestinal effects of the lack of Vanin-1 could be due to the differential expression of Vanin-1 and Vanin-3 genes, since both transcripts are present in the liver whereas only Vanin-1 is highly expressed by intestinal epithelial cells. Even though Vanin-3 is expressed in liver, Vanin-1^{-/-} mice lack free cysteamine (Pitari, 2000; Martin, 2001), which indicates that redundancy of vanin genes is not absolute.

2.b) Lack of Vanin-1 Prevented Intestinal Injury Following Indomethacin Treatment

The protective effect of Vanin-1 deletion has been explored in the context of acute inflammation. Histological examination of intestinal injury following indomethacin treatment showed that WT mice had flattened mucosal villi, which appeared swollen and increased breadthwise. At high magnification, it has been noted a large and clear space, which seemed to be due to a detachment of the lamina propria from the basement membrane of the villous epithelium, together with dilated lymphatic channels. In contrast, the intestine wall of the Vanin-1^{-/-} mice appeared normal, without flattened villi nor architectural desorganisation of the intestinal villi. These findings are in agreement with the high expression of Vanin-1 at the brush border of enterocytes in WT and not Vanin-1^{-/-} mice.

2.c) Lack of Vanin-1 Prevented Inflammatory Response to Indomethacin Treatment

In this indomethacin inflammatory model, the expression of a number of inflammatory factors has been evaluated following indomethacin treatment.

COX-1 was expressed in untreated WT and Vanin-1^{-/-} mice; the mRNA levels were not affected whatever the treatment applied. None of the other mRNAs (COX-2, iNOS and MIP-2-macrophage inflammatory protein-2) assayed were expressed under physiological conditions. Indomethacin treatment triggered a sharp augmentation of COX-2, iNOS and MIP-2 mRNAs in WT mice. In contrast, in Vanin-1^{-/-} mice COX-2 and iNOS mRNAs were barely amplified and MIP-2 mRNA was undetectable.

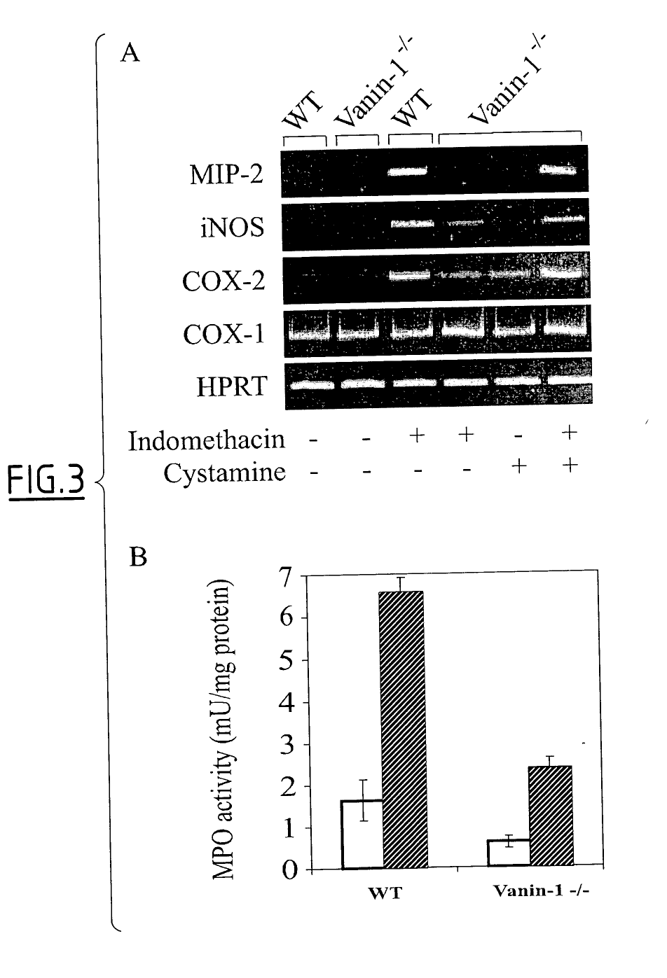

Intestinal myeloperoxidase (MPO) activity in response to S. mansoni infection and indomethacin treatment has further been assayed. Changes in mRNA expression of the neutrophil chemoattractant MIP-2 paralleled the activity of MPO. Thus, lower MIP-2 expression in Vanin-1^{-/-} was associated with a reduced MPO (myeloperoxidase) activity. Accordingly, enzyme activity was dramatically enhanced following infection with S. mansoni or treatment with indomethacin. In response to S. mansoni infection and indomethacin treatment, activity remained significantly lower in Vanin-1^{-/-} than in WT mice (FIG. 3B).

To link firmly these reduced inflammation in Vanin-1^{-/-} mice to cyst(e)amine deficiency, cystamine was given to indomethacin-treated mice. This treatment reversed the Vanin-1^{-/-} phenotype, i.e. restored mRNA expression to WT levels. The effect was not due to cystamine itself as, when administered without indomethacin, cystamine did not change iNOS and MIP-2 mRNAs, despite a moderate upregulation of COX-2 (FIG. 3A).

The results provided herein show that several transcripts associated with inflammatory cell activation, known to be induced by indomethacin (MacNaughton, 1998; Tanaka, 2002), were barely induced or undetectable in the intestine of Vanin-1^{-/-} mice. Among them is MIP-2, a local chemoattractant for neutrophils (Wolpe, 1989) plays an important role in the progression of indomethacin-induced intestinal inflammation (Beck, 2000). Absence of MIP-2 expression in Vanin-1 is associated with a reduced MPO activity. Therefore, the reduced intestinal injury in Vanin-1^{-/-} mice in respect with WT could be explained by the reduced recruitment of myeloid cells in the vicinity of colonic epithelial cells. In agreement, in man, the migratory function of neutrophils involves the Vanin cluster (Suzuki et al., 1999).

2.d) Lack of Vanin-1 Led to Increased GSH Levels in Liver and Intestine

Liver GSH stores were monitored (Table 1). They were significantly higher in Vanin-1^{-/-} than in WT mice, either in healthy animals or in experimental ones.

TABLE 1

| | WT | | KO | |
|---|---|---|---|---|
| | GSH | γ-GCS | GSH | γ-GCS |
| Controls | 8.56 ± 1.82 | 16.5 ± 2.2 | 12.7 ± 2.43* | 23.9 ± 3.43* |
| Cystamine | 5.36 ± 1.15 | 10.8 ± 0.63 | 5.55 ± 1.20 | 11.2 ± 0.58 |
| Indomethacin | 9.50 ± 1.44 | 23.4 ± 0.72 | 16.6 ± 1.26* | 37.9 ± 3.33* |
| Indomethacin + Cystamine | 7.60 ± 0.65 | 19.0 ± 4.12 | 8.30 ± 0.95 | 21.6 ± 1.71 |
| Infection with S. mansoni | 3.96 ± 1.8 | 20.6 ± 2.8 | 11.09 ± 1.85* | 37.2 ± 2.35* |

*values significantly different from the WT group;
P < 0.05, Student's t test.

Changes in GSH levels reflected changes in γ-GCS activity, which was in all cases significantly higher in Vanin-1^{-/-} than in WT mice. Enzyme activity increased following S. mansoni infection or indomethacin treatment, both conditions triggering an oxidative stress in the liver. However, enzyme activation was more pronounced in mice lacking Vanin-1 than in WT. In infected WT animals, liver GSH levels were lower than in WT controls, in spite of a enhanced γ-GCS activity; this is likely to result from the high level oxidative stress associated with schistosomiasis. By contrast, GSH levels did not significantly change in infected Vanin-1^{-/-} mice. Cystamine administration dramatically reduced both γ-GCS activity and GSH levels; this occurred in control as in indomethacin-treated animals. Importantly, cystamine suppressed the difference between Vanin-1^{-/-} and WT mice.

In the intestine (Table 2), the intrinsic γ-GCS activity showed little changes in Vanin-1^{-/-} vs WT mice, or in untreated indomethacin-treated animals.

TABLE 2

| | WT | | KO | |
|---|---|---|---|---|
| | GSH | γ-GCS | GSH | γ-GCS |
| Controls | 1.03 ± 0.13 | 26.5 ± 4.21 | 1.84 ± 0.21* | 23.6 ± 4.56 |
| | (2.4) | | | |
| Cystamine | 0.90 ± 0.12 | 20.2 ± 1.2 | 1.0 ± 0.05 | 21.5 ± 2.4 |
| Indomethacin | 0.56 ± 0.08 | 18.4 ± 2.18 | 1.37 ± 0.24* | 23.7 ± 2.63* |
| Indomethacin + Cystamine | 0.65 ± 0.06 | 17.7 ± 1.38 | 0.75 ± 0.08 | 20.6 ± 1.02 |
| Indomethacin + NAC | 0.79 ± 0.07 | 20.18 ± 1.24 | nd | nd |

*values significantly different from the WT group;
P < 0.05, Student's t test.

Enzyme activity was significantly higher in Vanin-1^{-/-} than in WT mice after indomethacin treatment; the difference was annihilated by administration of cystamine. In spite of little changes in γ-GCS activity, intestinal GSH levels paralleled those observed in liver, i.e. a dramatic increase in the absence of Vanin-1, in untreated as in indomethacin-treated mice. Moreover, as observed in liver, cystamine administration reduced intestinal GSH levels therefore annihilating the effects of Vanin-1 inactivation. Lastly, NAC administration to indomethacin-treated mice had no marked effect neither on γ-GCS activity nor on GSH levels.

Thus, the inventors showed that the reduced intestinal inflammation in Vanin-1^{-/-} mice was associated with increased GSH synthesis in liver. GSH, which is transferred from liver to other tissues, particularly the digestive tract (Hirota, 1989), plays a crucial regulatory function in the context of inflammation, as GSH is required to maintain the cellular redox status and to scavenge free radicals (Lu, 1999). Furthermore, GSH modulates immune functions via the regulation of stress-related transduction pathways and via the control of lipid mediator synthesis as a cofactor of glutathione peroxidases. The rate-limiting step for GSH synthesis is γ-GCS activity (Ikegami, 2000). γ-GCS mRNA expression and activity are increased as an adaptive response to oxidative stress (Lu, 1999). γ-GCS activity was increased following S. mansoni infection, with however a decrease of GSH concentration. In this case, GSH depletion is likely to be due to S. mansoni-induced oxidative processes in the liver (Gharib, 1999). In agreement with Sekhar (2002), the inventors found that indomethacin treatment increased γ-GCS activity and liver GSH content. They also found that, under physiological or inflamed conditions, γ-GCS activity was significantly higher in mice lacking the Vanin-1 gene, and GSH levels paralleled enzyme activity. Therefore Vanin-1^{-/-} mice better maintained GSH homeostasis in face of oxidative stress, preserving tissue integrity and function after inflammatory treatments.

2.e) Lack of Vanin-1 Led to Increased Thymic Reconstitution Following Damage by Irradiation

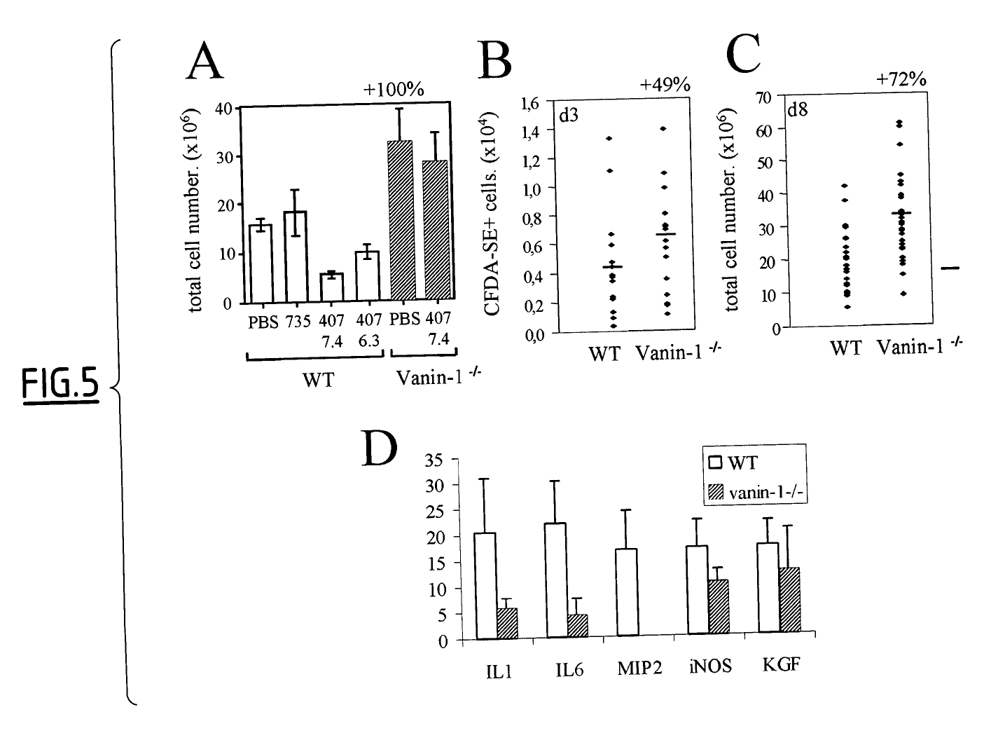

Anti-Vanin-1 mAbs subclones (407.6.3 and 407.7.4) inhibit thymic reconstitution in WT mice (FIG. 5A). Surprisingly, the phenotype observed with Vanin-1^{-/-} mice is the opposite that observed with the anti-Vanin-1 antibodies. Actually, the analysis of the cellularity in thymus 3 or 8 days post-irradiation showed an accelerated thymic reconstitution in Vanin-1^{-/-} mice as compared to WT animals (FIG. 5A). Indeed, there was in average 51% and 72% (FIG. 5C) more cells in thymuses of Vanin-1^{-/-} animals at day 3 or 8, respectively.

The thymic reconstitution assay was performed using intravenous injection of bone marrow cells previously labeled with the CFDA-SE fluorescent dye. Looking at the intrathymic fluorescent cells at day 3, an increase of 49% in the number of CFDA-SE+ cells (FIG. 5B) were found. These data led to conclude that Vanin-1^{-/-} mice display an accelerated thymic reconstitution that could be due, at least partially, to the entry of new bone marrow-derived cells and that characterize a better behavior of these animals in response to radiation injury.

2.f) Lack of Vanin-1^{-/-} is Associated With Better Response to Irradiation

In WT mice, ionizing irradiation induces the generation of ROS (radical oxygen species) in tissues and provokes an accumulation of macrophages with associated additional production of ROS and cytokines which is a key process involved in the physiopathology of radiation injury. In both post-irradiation models of thymus and intestine regeneration, the amount of activated macrophages was estimated by immunohistology using the tyramide staining as a substrate for the endogenous tissue peroxidase. There were less detectable activated macrophages in Vanin-1/thymus compared to WT control 24 hours post-irradiation.

Furthermore, RT-PCR was performed using several inflammatory and wound repair markers: IL1β, IL6, MIP2, INOS, IFNγ, COX1, COX2, KGF (Keratinocyte Growth Factor). All samples were standardized according to the amount of GAPDH and the obtained PCR products were quantified using the AIDA software (Advanced Image Data Analyzer AIDA1000/1D software 1.01. Raytest GmbH Germany). Among all markers tested IL1β, IL6, MIP2 and iNOS showed significant lower levels of transcripts in Vanin-1^{-/-} compared to WT mice thymus (FIG. 5D) and ileum while MIP2, iNOS and COX2 mRNAs showed lower levels in Vanin-1^{-/-} ileum.

These weaker inflammatory environment in Vanin-1^{-/-} post-irradiated tissues led to conclude that the lack of Vanin-1 is associated with a better resistance to oxidative stress induced by radiation in a process that might be linked to the inflammatory state of the tissue.

2.f) Lack of Vanin-1 Led to Increased GSH Levels in Liver and Thymus

Specific activities of the proteins involved in detoxifying cell system were assayed in thymus and liver homogenates from WT and Vanin-1^{-/-} mice, before and after γ-irradiation.

The following table (Table 3) shows that Vanin-1^{-/-} mice have a significantly higher γGCS activity in liver. As expected, this difference in γGCS activity was correlated to higher liver GSH stores whereas the level of oxidized GSSG and the recycling by GSH-Red appeared to be not significantly affected. Nevertheless, the γGCS activity and the GSH level were augmented in the WT mice following irradiation while they remained quite similar in the Vanin-1^{-/-} mice. Therefore, Vanin-1 is a modulator of the GSH pool at least acting on the γGCS activity through cysteamine.

TABLE 3
γGCS, GSH, GSSG and GSH-Red monitoring in WT and
Vanin-1^{-/-} mice either in healthy or in irradiated animals.

| | | | +γ-irradiation | |
|---|---|---|---|---|
| | WT | Vanin-1^{-/-} | WT | Vanin-1^{-/-} |
| γGCS | 32 ± 2.7 | 53 ± 3 | 46 ± 3 | 40 ± 4.4 |
| mU/mgprot | (n = 5) | (n = 5) | (n = 5) | (n = 5) |
| GSH | 6.04 ± 0.77 | 9.00 ± 2.73 | 9.80 ± 2.99 | 13.5 ± 2.51 |
| μmol/g. liver | (n = 6) | (n = 5) | (n = 4) | (n = 4) |
| GSSG | 0.18 ± 0.05 | 0.11 ± 0.02 | 0.20 ± 0.03 | 0.19 ± 0.03 |
| μmol/g. liver | (n = 7) | (n = 3) | (n = 4) | (n = 4) |
| GSH-Red | 27 ± 3.5 | 29 ± 1.3 | 24 ± 2.2 | 19 ± 1.78 |
| mU/mg prot | (n = 5) | (n = 5) | (n = 5) | (n = 5) |

n = number of animals

The following table (Table 4) shows that no significant differences were observed in the superoxide dismutase (SOD) and catalase (CAT) specific enzymatic activities comparing WT and Vanin-1^{-/-} organs. However, irradiation produced a slightly more pronounced decrease of the SOD activity in Vanin-1^{-/-} tissues. Whereas the glutathione S-transferase (GST) activity was comparable between both genotypes in liver, thymus showed a divergent feature with a faintly lower GST activity in Vanin-1^{-/-} thymus under physiological conditions but a marked augmentation after irradiation.

TABLE 4
SOD, CAT and GST activities in WT and Vanin-1^{-/-} mice either in healthy or in irradiated animals.

| | | | γ-irradiation | | | | γ-irradiation | |
|---|---|---|---|---|---|---|---|---|
| | LIVER | | | | THYMUS | | | |
| | WT | Vanin-1^{-/-} | WT | Vanin-1^{-/-} | WT | Vanin-1^{-/-} | WT | Vanin-1^{-/-} |
| SOD | 8700 ± 1400 | 11358 ± 460 | 8238 ± 948 | 5690 ± 682 | 34556 ± 487 | 38816 ± 2300 | 12237 ± 1732 | 8072 ± 565 |
| CAT | 450 ± 85 | 462 ± 33.5 | 342 ± 40 | 277 ± 14 | 14685 ± 1300 | 16256 ± 1385 | 16385 ± 130 | 15828 ± 681 |
| GST | 1676 ± 121 | 1800 ± 178 | 1193 ± 72 | 1000 ± 106 | 66 ± 3 | 47 ± 4 | 54 ± 1.4 | 86 ± 18 |

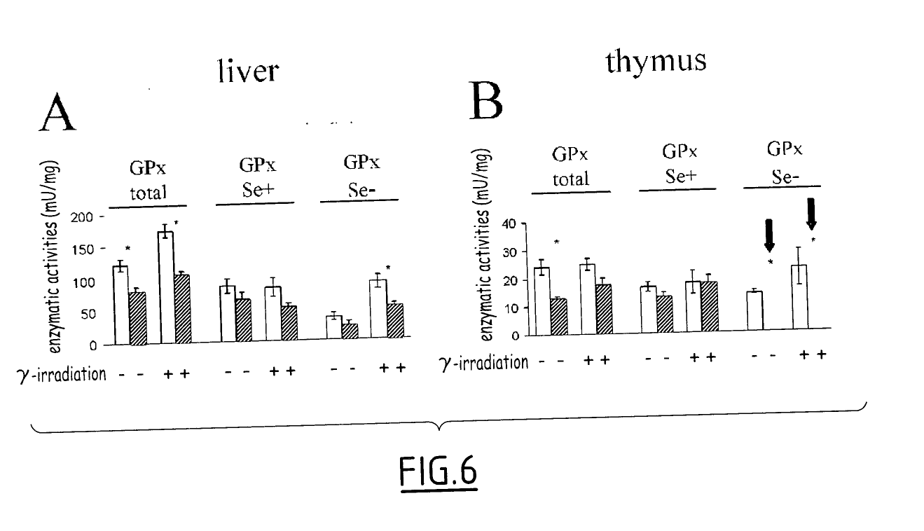

In contrast, the total glutathione peroxidase (GPx) activity was found significantly decreased in the absence of Vanin-1 in both thymus and liver (FIG. 6). Glutathione peroxidases catalyse the: reduction of peroxides in the cells. Several enzymes display GPx activity and has been classified as selenium-dependent (Se+) or selenium-independent (Se−). Whereas GPx-Se+ can reduce H₂O₂, GPx-Se− activity which is correlated to multiple enzymes from the GST class, is inactive with H₂O₂ and exhibit only activity with organic hydroperoxides. As shown in FIG. 6, the lower level detected for the total GPx activity in Vanin-1^{-/-} tissues is attributable to an impaired Se-independent activity belonging to some classes of GSTs. This was more severe in thymus where the GPx-Se− activity was undetectable. In addition this reduced GPx-Se-activity was not rescued after irradiation-induced oxidative insult. Thus, Vanin-1 might exert a control at the GST level influencing the cellular GPx-Se-detoxifying response.

Vanin-1^{-/-} mice which lack cysteamine show a higher γGCS activity and consequently elevated GSH stores in tissues. Strikingly, the lack of Vanin-1 is associated with an antioxidant state at physiological conditions which is comparable to those of the WT mice following irradiation; such as the Vanin-1^{-/-} tissues were better armed to respond to oxidative harm.

2.g) Lack of Vanin Prevented TNBS-Induced Colitis, a Th1 Based Model of Inflammatory Bowel (Crohn's) Disease

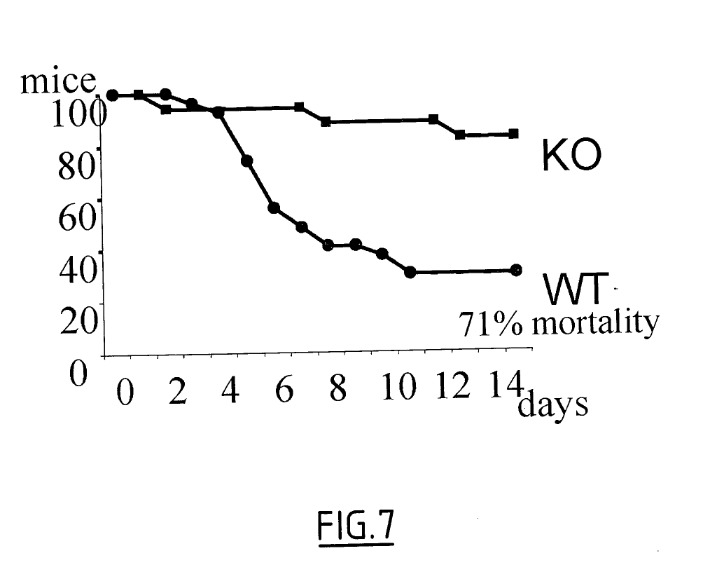

FIG. 7 shows that Vanin-1 deficient mice significantly resist TNBS-induced colitis, as compared to wild-type mice.

Furthermore, the survival of the Vanin-1^{-/-} mice is associated with a reduced inflammation and necrosis of the intestine.

### REFERENCES

Abdallahi, O. M., Hanna, S., De Reggi, M. & Gharib, B. (1999) Liver 19, 495-500.

Aki S, Caillaud C, Vigne E, Strafford-Perricaudet L D, Poenaru L, Perricaudet M, Kahn A, Peschanski M R. (1993) Nat Genet 3, 224-8.

Aurrand-Lions, M., Galland, F., Bazin, H., Zakharyev, V., Imhof, B. A. & Naquet, P. (1996) Immunity 5, 391-405.

Ballou, L. R., Botting, R. M., Goorha, S., Zhang, J. & Vane, J. R. (2000) Proc Natl Acad Sci USA 97, 10272-6.

Barbas C F 3rd, Bain J D, Hoekstra D M, Lerner R A. (1992) Proc Natl Acad Sci USA; 89, 4457-61.

Beamer, R. L., Griffith, O. W., Gass, J. D., Anderson, M. E. & Meister, A. (1980) J Biol Chem 255, 11732-6.

Beck, P. L., Xavier, R., Lu, N., Nanda, N. N., Dinauer, M, Podoisky, D. K. & Seed, B. (2000) Gastroenterology 119, 699-705.

Brummelkamp T. R. (2002) Science 296, 550-553

Cech T R. (1989) Biochem Int 18, 7-14.

Cheever, A. W. (1985) Exp Parasitol 59, 1-11.

Chiocca E A, Choi B B, Cai W Z, DeLuca N A, Schaffer P A, DiFiglia M, Breakefield X O, Martuza R L. (1990) New Biol 2, 739-46.

Colas P, Cohen B, Jessen T, Grishina I, McCoy J, Brent R. (1996) Nature 380, 548-50.

Danos O, Mulligan R C. (1988) Proc Natl Acad Sci USA 85, 646-04.

Desreumaux P, Dubuquoy L, Nutten S, Peuchmaur M, Englaro W, Schoonjans K, Derijard B, Desvergne B, Wahli W, Chambon P, Leibowitz M D, Colombel J F, Auwerx J. (2001) J. Exp. Med. 193, 827-838.

Dobson A T, Margolis T P, Sedarati F, Stevens J G, Feldman L T. (1990) Neuron 5, 353-60.

Dupre, S., Graziani, M. T., Rosei, M. A., Fabi, A. & Del Grosso, E. (1970) Eur J Biochom 16, 571-8.

Ernest, M. J. & Kim, K. H. (1973) J Biol Chem 248, 1550-5

Felgner P L, Gadek T R, Holm M, Roman R, Chan H W, Wenz M, Northrop J P, Ringold G M, Danielsen M. (1987) Proc Natl Acad Sci USA 84, 7413-7.

Feigner P L, Ringold G M. (1989) Nature 337, 387-8.

Fraley R, Subramani S, Berg P, Papahadjopoulos D. J (1980) Biol Chem 255, 10431-5.

Funk C. D. & Chen X. S. (2000) Am J Respir Crit Care Med 161, 120-124.

Galland, F., Malergue, F., Bazin, H., Mattei, M. G., Aurrand-Lions, M., Theillet, C. & Naquet, P. (1998) Genomics 53, 203-13.

Gharib, B., Abdallahi, O. M., Dessein, H. & De Reggi, M. (1999) J Hepatol 30, 594-602.

Graham F L, Smiley J, Russell W C, Nairn R. (1977) J Gen Virol 36, 59-74.

Graham F L. (1984) EMBO J 3, 2917-22.

Granjeaud, S., Naquet, P. & Galland, F. (1999) Immunogenetics 49, 964-72.

Griffith, O. W., Larsson, A. & Meister, A. (1977) Biochem Biophys Res Commun 79, 919-25.

Haddad J. J. (2002) Biochem Pharmacol. 63, 305-20

Harlow E. et al., Antibodies: A Laboratory Manual, Cold Spring Harbor Laboratory, New York, (1988)

Heemskerk M. H. M. 1999 Cell Immunol 195, 10-17

Hirota, M., Inoue, M., Hirayama, K., Morino, Y., Sakamoto, K., Mori, K. & Akagi, M. (1989) Gastroenterology 97, 853-9

Ikegami, Y., Tatebe, S., Lin-Lee, Y. C., Xie, Q. W., Ishikawa, T. & Kuo. M. T. (2000) J Cell Physiol 185, 293-301

Jayasena S D. (1999) Clin Chem 45, 1628-50.

Kaneda Y, Iwai K, Uchida T. (1989) Science 243, 375-8.

Kerneis S., Bogdanova A., Kraehenbuhl J. P., Pringault E. (1997) Science 277 949-52.

Kohler G and Milstein C. (1975) Nature 256, 4957.

Konecki, D. S., Brennand, J., Fuscoe, J. C., Caskey, C. T. & Chinault, A. C. (1982) Nucleic Acids Res 10, 6763-75.

La Flamme, A. C., Patton, E. A., Bauman, B. & Pearce, E. J. (2001) J Immunol 166, 1903-11.

Lambertucci, J. R., Serufo, J. C., Gerspacher-Lara, R., Rayes, A. A., Teixeira, R., Nobre, V. & Antunes, C. M. (2000) Acta Trop 77, 101-9.

Le Gal La Salle G, Robert J J, Berrard S, Ridoux V, Stratford-Perricaudet L D, Perricaudet M, Mallet J. (1993) Science 259, 988-90

Lebo, R. V. & Kredich, N. M. (1978) J Biol Chem 253, 2615-23.

Levrero M, Barban V, Manteca S, Ballay A, Balsamo C, Avantaggiati M L, Natoli G, Skeliekens H, Tiollais P, Perricaudet M. (1991) Gene 101, 195-202.

Levrero M, Barban V, Manteca S, Ballay A, Balsamo C, Avantaggiati M L, Natoli G, Skellekens H. Tiollais P, Perricaudet M. (1991) Gene 101, 195-202.

Liaudet, L., Soriano, F. G., Szabo, E. Virag, L., Mabley, J. G., Salzman, A. L. & Szabo, C. (2000) Proc Natl Acad Sci USA 97, 10203-8.

Lu, S. C. (1999) Faseb J 13, 1169-83

Lyons, C. R., Orloff, G. J. & Cunningham, J. M. (1992) J Biol Chem 267, 63704.

MacNaughton, W. K., Lowe, S. S. & Cushing, K. (1998) Am J Physiol 275, G1353-60.

Mannick J B, & Schonhoff C M. (2002) Arch Biochem Biophys 408, 1-6.

Maras, B., Barra, D., Dupre, S. & Pitari, G. (1999) FEBS Lett 461, 149-52.

Martin, F., Malergue, F., Pitari, G., Philippe, J. M., Philips, S., Chabret, C., Granjeaud, S., Mattei, M. G., Mungall, A. J., Naquet, P. & Galland, F. (2001) Immunogenetics 53, 296-306.

Miller, S. L. & Schlesinger, G. (1993) J Mol Evol 36, 302-7.

Miyanohara A, Johnson P A, Elam R L, Dai Y, Witztum J L, Verma I M, Friedmann T. (1992) New Biol 4, 238-46.

Namboodiri, M. A., Weller, J. L. & Klein D. C. (1980) Biochem Biophys Res Commun 96, 188-95.

Neurath M, Fuss I, Strober W. (2000) Int Rev Immunol 19:51-62.

Ohtsuka Y, Lee J, Stamm D S, Sanderson I R (2001) Gut 49, 526-533.

Orth T, Neurath M, Schirmacher P, Galle P R, Mayet W J. (2000) J Hepatol, 33, 862-72.

Pagano J S, McCutchan J H, Vaheri A. (1967) J Virol. 1, 891-7.

Pascal, M., Abdallahi, O. M., Elwali, N. E., Mergani, A., Qurashi, M. A., Magzoub, M., de Reggi, M. & Gharib, B. (2000) Trans R Soc Trop Med Hyg 94, 66-70.

Pitari G, Maurizi G, Ascenzi P, Ricci G, Dupre S. (1994) Eur J Biochem 15;226(1):81-6.

Pitari, G., Malergue, F., Martin, F., Philippe, J. M., Massucci, M. T., Chabret, C., Maras, B., Dupre, S., Naquet, P. & Galland, F. (2000) FEBS Lett 483, 149-54.

Pontremoli, S., Traniello, S., Enser, M., Shapiro, S. & Horecker, B. L. (1967) Proc Natl Acad Sci USA 58, 286-93

Ricci, G., Nardini, M., Chiaraluce, R., Dupre, S. & Cavallini, D. (1983) J Appl Biochem 5, 320-9.

Roemer K, Johnson P A, Friedmann T. (1992) J Gen Virol 73, 1553-8.

Rubin-Carrez C. (2000). Biofutur, 199

Sambrook et al. (1989) Molecular cloning, a laboratory manual Spring Harbor Laboratory Press, 9.54-62

Scott, D. J., Hull, M. A., Cartwright, E. J., Lam, W. K., Tisbury, A., Poulsom, R., Markham, A. F., Bonifer, C. & Coletta, P. L. (2001) Gastroenterology 121, 889-99.

Seelig, G. F. & Meister, A. (1984) Anal Biochem 141, 510-4.

Sekhar, K. R., Spitz, D. R., Harris, S., Nguyen, T. T., Meredith, M. J., Holt, J. T., Gius, D., Marnett, L. J., Summar, M. L., Freeman, M. L. & Guis, D. (2002) Free Radic Biol Med 32, 650-62.

Sharp P A. (2001) Genes Dev 15, 485-90.

Siefring, G. E., Jr., Apostol, A. B., Velasco, P. T. & Lorand, L. (1978) Biochemistry 17, 2598-604.

Smith L. L., (1987) Human Toxicol 6, 3136.

Smith W. L., DeWitt D. L., Garavito R. M. (2000) Annu. Rev Biochem. 69, 145-182.

Stratford-Perricaudet L D, Levrero M, Chasse J F, Perricaudet M, Briand P. (1990) Hum Gene Ther 1, 241-56.

Suzuki, K., Watanabe, T., Sakurai, S., Ohtake, K., Kinoshita, T., Araki, A., Fujita, T., Takei, H., Takeda, Y., Sato, Y., Yamashita, T., Araki, Y. & Sendo, F. (1999) J Immunol 162, 4277-84.

Tanaka, A., Araki, H., Hase, S., Komoike, Y. & Takeuchi, K. (2002) Aliment Pharmacol Ther 16, 90-101.

Tietze, F. (1969) Anal Biochem 27, 502-22. La Flamme, A. C., Patton, E. A., Bauman, B. & Pearce, E. J. (2001) J Immunol 166, 1903-11.

Tuerk C. and Gold. L. (1990) Systematic evolution of ligands by exponential enrichment: RNA ligands to bacteriophage T4 DNA polymerase. Science. 3;249(4968):505-10.

Van Vliet E., Melis M., van Ewijk W. (1984) 87, 101-9

Waterhouse P, Griffiths A D, Johnson K S, Winter G. (1993) Nucleic Acids Res 21, 2265-6.

Wilson J M, Grossman M, Wu C H, Chowdhury N R, Wu G Y, Chowdhury J R. (1992) J. Biol. Chem. 267, 963-967.

Wolpe S. D. & Cerami A. (1989) Faseb J 3, 2565-73.

Wu G Y, Wu C H. (1988) J. Biol. Chem. 263, 14621-14624.

Wynn, T. A., Cheever, A. W., Williams, M. E., Hieny, S., Caspar, P., Kuhn, R., Muller, W. & Sher, A. (1998) J Immunol 160, 4473-80.

Yotnda P. Onishi H, Heslop H E, Shayakhmetov D, Lieber A, Brenner M, Davis A. (2001) Gene Ther 8, 930-937.

1. A method for identifying a compound useful for the treatment of an inflammatory and/or an oxidative disorder, comprising the steps consisting of determining the capacity of a test compound having Vanin-1 antagonist activity to prevent or inhibit an induced inflammation and/or oxidative disorder or state.

2. The method according to claim 1, comprising the steps consisting of:
a) administering a mammal with (i) an inducer of an inflammatory and/or oxidative disorder, in a amount suitable for inducing inflammatory and/or an oxidative disorder in said mammal and (ii) a test compound; and
b) determining the capacity of the test compound to prevent or inhibit the inflammatory and/or oxidative disorder in said mammal, wherein a reduced inflammation and/or oxidative disorder, or a non-appearance thereof, is indicative of a compound useful for the treatment of an inflammatory and/or oxidative disorder.

3. The method according to claim 1, comprising the steps consisting of:
a) contacting a gut sample from a mammal with an inducer of an inflammatory and/or oxidative disorder, and a test compound;
b) determining the capacity of the test compound to prevent or inhibit the inflammatory and/or oxidative disorder induced in gut, wherein a reduced inflammatory and/or oxidative disorder, or a non-appearance thereof, is indicative of a compound useful for the treatment of an inflammatory and/or oxidative disorder.

4. The method according to claim 1, wherein the test compound is administered prior to the inducer of an inflammatory and/or oxidative disorder, and the inflammatory and/or oxidative disorder of the mammal or gut sample is assessed before and after each administration, and compared with the inflammatory and/or oxidative disorder of a control mammal or gut sample to which a control vehicle has been administered in lieu of the test compound;
wherein a reduced inflammatory and/or oxidative disorder, or non-appearance thereof, with comparison to the control mammal or gut sample is indicative of a test compound having Vanin-1 antagonist activity useful for the treatment of an inflammatory and/or oxidative disorder.

5. The method according to claim 1, wherein the test compound is administered after the inducer of an inflammatory and/or oxidative disorder, and the inflammatory and/or oxidative disorder of the mammal or gut sample is assessed before and after each administration, and compared with the inflammatory and/or oxidative disorder of a control mammal or gut sample to which a control vehicle has been administered or contacted in lieu of the test compound;
wherein a reduced inflammatory and/or oxidative disorder with comparison to the control mammal or gut sample is indicative of a test compound having Vanin-1 antagonist activity useful for the treatment of an inflammatory and/or oxidative disorder.

6. The method according to claim 2, wherein said inducer of an inflammatory and/or oxidative disorder is selected from the group consisting of a non-steroidal anti-inflammatory drug (NSAID), trinitrobenzenesulphonic acid (TNBS), Schistosoma mansoni, H₂O₂, t-butyl-hydroquinone (TBHQ) and irradiation.

7. The method according to claim 2, wherein said mammal is selected from the group consisting of a mouse, a rat, a monkey, a dog, and a cat.

8. The method according to claim 1, comprising the steps consisting of:
a) contacting a cell with an inducer of an inflammatory and/or oxidative state, and a test compound;
b) determining the capacity of the test compound to prevent or inhibit the inflammation and/or oxidative state induced in said cell, wherein a reduced inflammation and/or oxidative state, or a non-appearance thereof, is indicative of a compound useful for the treatment of an inflammatory and/or oxidative disorder.

9. The method according to claim 8, wherein the test compound is contacted with the cell prior to the inducer of an inflammatory and/or oxidative state, and the inflammatory and/or oxidative state of the cell may be assessed before and after each contacting, and compared with the inflammatory and/or oxidative state of a control cell that has been contacted with a control vehicle in lieu of the test compound;
wherein a reduced inflammatory and/or oxidative state, or non-appearance thereof, with comparison to the control cell is indicative of a test compound having Vanin-1 antagonist activity useful for the treatment of an inflammatory and/or oxidative disorder.

10. The method according to claim 8, wherein the test compound is contacted with the cell after the inducer of an inflammatory and/or oxidative state, and the inflammatory and/or oxidative condition of the cell may be assessed before and after each contacting, and compared with the inflammatory and/or oxidative state of a control cell that has been contacted with a control vehicle in lieu of the test compound;
wherein, a reduced inflammatory and/or oxidative state with comparison to the control cell is indicative of a test compound having Vanin-1 antagonist activity useful for the treatment of an inflammatory and/or oxidative disorder.

11. The method according to claim 8, wherein the inducer of an inflammatory and/or oxidative state is selected from the group consisting of IL-1, IFN, Paraquat, H₂O₂, TBHQ, irradiation.

12. The method according to claim 8, wherein said cell is an epithelial cell.

13. The method according to claim 12, wherein said cell is an epithelial cell from the gastrointestinal tract.

14. The method according to claim 8, wherein the inflammatory disorder is selected from the group consisting of Crohn's disease and an ulcer resulting from administration of a non-steroidal anti-inflammatory drug.

15. A method for the treatment of an inflammatory and/or an oxidative disorder that comprises administering a patient in need thereof with therapeutically effective amount of a Vanin-1 antagonist, in a pharmaceutically acceptable carrier.

16. The method according to claim 15, wherein the Vanin-1 antagonist is an antibody capable of specifically interacting with Vanin-1 and inhibiting its activity.

17. The method according to claim 15, wherein the Vanin-1 antagonist is a nucleic acid blocking transcription of Vanin-1 gene and/or translation of Vanin-1 mRNA.

18. The method according to claim 15, wherein said inflammatory disorder is a gastrointestinal inflammatory disorder.

19. The method according to claim 15, wherein said inflammatory disorder is Crohn's disease.

20. The method according to claim 15, wherein said inflammatory disorder is an ulcer resulting from a non-steroidal anti-inflammatory drug treatment.
